(19) United States
(12) Patent Application Publication
(10) Pub. No.: US 2022/0349896 A1
(43) Pub. Date: Nov. 3, 2022

(54) METHODS OF EVALUATING POLYPEPTIDE-MODIFIED POLYMERS IN COMPOSITIONS

(71) Applicant: SIGILON THERAPEUTICS, INC., Cambridge, MA (US)

(72) Inventors: Richard Heidebrecht, Somerville, MA (US); Zoe Yin, Boston, MA (US)

(21) Appl. No.: 17/764,096

(22) PCT Filed: Sep. 25, 2020
(86) PCT No.: PCT/US2020/052870

§ 371 Date: Mar. 25, 2022

Related U.S. Application Data
(60) Provisional application No. 62/907,391, filed on Sep. 27, 2019.

(51) Int. Cl. G01N 33/68; G01N 1/40; G01N 1/44; C07K 19/00; G01N 30/86; G01N 30/88

(52) U.S. Cl. CPC G01N 33/6848; G01N 1/4044; G01N 1/44; C07K 19/00; G01N 30/8631; G01N 30/88; G01N 2030/8831; G01N 2030/027

(57) ABSTRACT

Described herein are methods for evaluating polymer compositions comprising polymers modified with a polypeptide (e.g., a cell-binding polypeptide), including methods for determining polypeptide concentration.

### BACKGROUND

Treating chronic and genetic diseases by implanting cells engineered to produce a therapeutic substance capable of treating such diseases has exciting potential to improve the health of patients with such diseases. To fully achieve this potential, the implanted cells must be protected from the patient's immune response, so that they remain viable, and the implanted cells must also be capable of producing therapeutic levels of the desired therapeutic substance for several weeks, months, or even longer. One approach for delivering such cellular therapies is to encapsulate the engineered cells into semi-permeable devices (e.g., hydrogel capsules), with the objective that the device structure isolates the cells from host immune system cells while allowing entry of nutrients for the cells and exit of the produced therapeutic substances. In some cases, these semi-permeable devices (e.g., hydrogel capsules) may contain one or more polymers modified with various substances, including polypeptides. A need exists to improve methods of evaluating these semi-permeable devices (e.g., hydrogel capsules), for example, by quantifying the polypeptide concentration levels bound to the devices, which can affect the viability and/or productivity of living cells contained within.

### SUMMARY

The present disclosure provides, at least in part, methods for evaluating polymer composition comprising a polymer modified with a polypeptide. In an embodiment, the evaluating comprises determining the concentration of the polypeptide within the polymer composition. The polymer composition may be used to prepare semi-permeable devices (e.g., hydrogel capsules) that comprise at least one compartment encapsulating a plurality of cells capable of expressing a therapeutic agent, which may be released from the device, e.g., upon implantation in a subject. In an embodiment, the polypeptide bound to the modified polymer may increase the viability of the cells and/or increase productivity of the cells in vitro (e.g., when the device is incubated in a cell culture medium) or in vivo (e.g., when the device is implanted in or otherwise administered to a subject).

Without wishing to be bound by theory, the concentration of certain polypeptides (e.g., cell-binding polypeptides) within polymer compositions used to prepare a semi-permeable device can have a significant impact on the viability of the encapsulated cells and/or levels of a therapeutic agent produced by and/or released from the encapsulated cells. As such, it may be useful to determine the concentration of such polypeptides in the polymer composition in order to, for example, modulate cell activity and viability. However, current methods for quantifying polypeptide concentrations are not ideal for use in evaluating the polymer compositions described herein. For example, the polypeptides described herein may not comprise a chromophore or other signature feature to detect in a concentration assay. In addition, the complexity of the modified polymers within a polymer composition can hamper efforts to achieve a reliable concentration value for the modifying polypeptide. The methods described herein are designed to circumvent these obstacles and provide the polypeptide concentration of a modified polymer within a polymer composition in a reliable manner.

In one aspect, the present disclosure features a method of evaluating a polymer composition comprising a polymer modified with polypeptide, the method comprising (a) subjecting the polymer composition to reaction conditions that allow for (i) release of the polypeptide from the modified polymer; and/or (ii) hydrolysis of the polypeptide into component amino acids. In an embodiment, the method comprises release of the polypeptide from the modified polymer and hydrolysis of the polypeptide into component amino acids. In an embodiment, the method comprises hydrolysis of the polypeptide into component amino acids. In an embodiment, the method comprises release of the polypeptide from the modified polymer. The method may further comprise (b) acquiring a value for the concentration of each component amino acid of the polypeptide; and (c) using the value obtained in step (b), acquiring a value for the concentration of the polypeptide bound to the modified polymer. In an embodiment, the modified polymer is a modified polysaccharide. In an embodiment, the polymer in the modified polymer (e.g., the polymer used to prepare the modified polymer) is an alginate (e.g., having an average molecular weight of 75 kD to 150 kD and/or a guluronate to mannuronate (G:M) ratio of greater than or equal to 1.5).

The polypeptide may be covalently bound or non-covalently bound to the modified polymer (e.g., modified alginate). In some embodiments, the polypeptide is covalently bound to the modified polymer (e.g., modified alginate), e.g., through a linker (e.g., an amino acid linker). In an embodiment, the polypeptide comprises a cell-binding polypeptide (CBP). Exemplary CBP sequences include RGD, RGDSP, DGEA, FYFDLR, PHSRN, YIGSR, and the sequences listed in Table 1, described herein. In an embodiment, the CBP comprises RGD. In an embodiment, the CBP comprises RGDSP. In an embodiment, the CBP comprises DGEA. In an embodiment, the CBP comprises FYFDLR. In an embodiment, the CBP comprises PHSRN. In an embodiment, the CBP comprises YIGSR.

In some embodiments, the reaction conditions that allow for release of the polypeptide from the modified polymer comprise contacting the polymer composition with an acidic solution, a basic solution, an enzymatic solution, light, microwave irradiation, heat, or a combination thereof. In some embodiments, the reaction conditions that allow for hydrolysis of the polypeptide into component amino acids comprise contacting the polymer composition device with an acidic solution, a basic solution, an enzymatic solution, light, microwave irradiation, heat, or a combination thereof. In an embodiment, the release of the polypeptide from the modified polymer and the hydrolysis of the polypeptide into component amino acids are achieved in the same step. In an embodiment, the release of the polypeptide from the modified polymer and the hydrolysis of the polypeptide into component amino acids are achieved in sequential steps or under different conditions.

In an embodiment, acquiring a value for the concentration of each component amino acid of the polypeptide bound to the modified polymer is achieved through a separation step. Exemplary separation steps include filtration, electrophoresis, or chromatography (e.g., size-exclusion chromatography, ion-exchange chromatography, gel filtration chromatography, reversed-phase chromatography, or hydrophobic interaction chromatography). In some embodiments, the individual amino acids are modified prior to separation, e.g., by derivatizing with a detection agent. In an embodiment, acquiring the concentration of each component amino acid of the polypeptide entails comparison of the level of each component amino acid with a standard.

In an embodiment, upon obtaining a value for the concentration of each individual amino acid, the concentration of polypeptide bound to the modified polymer is acquired by comparing the concentration of each component amino acid with the known sequence of the peptide or polypeptide. In an embodiment, the method further comprises acquiring the concentration of unconjugated polypeptide (i.e., free polypeptide) in the polymer composition.

In another aspect, the present disclosure features a method of evaluating a polymer composition comprising a polymer modified with a polypeptide, wherein the method does not comprise subjecting the polymer compositions to reaction conditions that allow for release of the polypeptide from the modified polymer. In an embodiment, the modified polymer within the polymer compositions are intact. In an embodiment, the modified polymer within the polymer composition is subjected to non-degradative conditions. In an embodiment, the method comprises acquiring a value of the total polypeptide conjugated (e.g., covalently bound) to a polymer in the polymer composition; and using the values obtained to acquire a value for the concentration of the polymer modified with a polypeptide, thereby evaluating the polymer composition. In an embodiment, the modified polymer is a modified polysaccharide. In an embodiment, the polymer in the modified polymer composition (e.g., the polymer used to prepare the modified polymer) is an alginate (e.g., having an average molecular weight of 75 kD to 150 kD and/or a guluronate to mannuronate (G:M) ratio of greater than or equal to 1.5). In an embodiment, the polypeptide comprises a cell-binding polypeptide (CBP), e.g., a sequence selected from RGD, RGDSP, DGEA, FYFDLR, PHSRN, YIGSR, and a sequence listed in Table 1. In an embodiment, the method comprises acquiring a value for the refractive index of the polymer composition. In an embodiment, acquiring the value of the refractive index comprises acquiring a refractometer reading (nD) at a specific wavelength and/or specific temperature.

In another aspect, the present disclosure features modified polymers and polymer compositions comprising a concentration of polypeptide between 0.05 umol/g and 2 umol/g, e.g., as determined by % weight of polymer (e.g., % weight of modified polymer), based on a method described herein. In an embodiment, the concentration of polypeptide bound to a modified polymer is between 0.1 umol/g and 1.0 umol/g, 0.2 umol/g and 0.8 umol/g, or 0.3 umol/g and 0.6 umol/g, e.g., as determined by % weight of polymer, based on a method described herein.

The details of one or more embodiments of the disclosure are set forth herein. Other features, objects, and advantages of the disclosure will be apparent from the Detailed Description, the Figures, the Examples, and the Claims.

### BRIEF DESCRIPTION OF DRAWINGS

FIGS. 1A-1B depict exemplary HPLC chromatograms acquired in the determination of the level of total CBP in a sample of CBP-modified alginate, as outlined in Example 2. FIG. 1A shows the UV chromatogram corresponding to the standard solution, while FIG. 1B shows a fluorescence chromatogram corresponding to the total CBP solution.

FIGS. 2A-2B depict exemplary HPLC chromatograms acquired in the determination of the level of unconjugated (e.g., free) CBP in a sample of CBP-modified alginate, as outlined in Example 3. FIG. 2A shows a chromatogram corresponding to the standard solution, while FIG. 2B shows a chromatogram corresponding to the free CBP solution.

### DETAILED DESCRIPTION

The present disclosure features methods for evaluating polymer compositions, wherein the polymer compositions comprise at least one polymer modified by a polypeptide, such as a cell binding peptide (CBP). The polymer compositions may be used to encapsulate a plurality of cells (e.g., live cells) capable of expressing a therapeutic agent when the device is implanted into a subject (e.g., a human). In an embodiment, the device further comprises a means for mitigating the foreign body response (FBR) when implanted into the subject.

In an embodiment, the polymer compositions are evaluated by quantifying the concentration of a polypeptide bound to a modified polymer in the polymer composition. The peptide may be covalently bound or non-covalently bound to the polymer(s) within the polymer composition. The concentration the bound polypeptide may be evaluated, for example, by acquiring the total concentration of the polypeptide (bound and unbound) in a first sample of the polymer composition (i.e., the “total polypeptide”), acquiring the concentration of unbound polypeptide (i.e., the “free polypeptide), and subtracting the free polypeptide from the total polypeptide. In another embodiment, the polymer composition is evaluated by acquiring a value of the refractive index of the polymer composition. The polymer composition may or may not be subjected to conditions to hydrolyze the polypeptide from the modified polymer. Methods for determining the polypeptide concentration within polymer compositions are described herein.

### Abbreviations and Definitions

Throughout the detailed description and examples of the disclosure the following abbreviations will be used:
CBP cell-binding polypeptide
CBP-polymer polymer covalently modified with a CBP via a linker
CM-Alg chemically modified alginate
CM-LMW-Alg chemically modified, low molecular weight alginate
CM-HMW-Alg chemically modified, high molecular weight alginate
CM-MMW-Alg chemically modified, medium molecular weight alginate
HMW-Alg high molecular weight alginate
MMW-Alg medium molecular weight alginate
RGD-alginate an alginate covalently modified with a peptide comprising the amino acid sequence RGD
U-Alg unmodified alginate
U-HMW-Alg unmodified high molecular weight alginate
U-LMW-Alg unmodified low molecular weight alginate
U-MMW-Alg unmodified medium molecular weight alginate
70:30 CM-Alg:U-Alg 70:30 mixture (V:V) of a chemically modified alginate and an unmodified alginate, e.g., as described in Example 2

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” means when used herein to modify a numerically defined parameter (e.g., a physical description of a hydrogel capsule such as diameter, sphericity, number of cells encapsulated therein, the number of capsules in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. In some embodiments, the term “about” means that the modified parameter may vary by as much as 15%, 10% or 5% above and below the stated numerical value for that parameter. Alternatively, particularly with respect to certain properties of the polymer compositions described herein, such as concentration of a polypeptide bound to a modified polymer, the term “about” can mean within an order of magnitude above and below the recited value, e.g., within 5-fold, 4-fold, 3-fold, 2-fold or 1-fold.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data or using a refractometer to acquire a refractive index value.

“Administer”, “administering”, or “administration”, as used herein, refer to implanting, absorbing, ingesting, injecting or otherwise introducing into a subject, an entity described herein (e.g., a device or a preparation of devices), or providing such an entity to a subject for administration.

“Afibrotic”, as used herein, means a compound or material that mitigates the foreign body response (FBR). For example, the amount of FBR in a biological tissue that is induced by implant into that tissue of a device (e.g., hydrogel capsule) comprising an afibrotic compound is lower than the FBR induced by implantation of an afibrotic-null reference device, i.e., a device that lacks any afibrotic compound, but is of substantially the same composition (e.g., same CBP-polymer, same cell type(s)) and structure (e.g., size, shape, no. of compartments).

“Cell,” as used herein, refers to an engineered cell or a cell that is not engineered. In an embodiment, a cell is an immortalized cell or an engineered cell derived from an immortalized cell. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). In an embodiment, the CBP is capable of mimicking at least one activity of a ligand for a cell-adhesion molecule (CAM) or other cell-surface molecule that mediates cell-matrix junctions or cell-cell junctions or other receptor-mediated signaling. The CBP may be less than 250, 200, 150, 100, 75, 50, 40 30, 25, 20, 15 or 10 amino acids in length. In an embodiment, the CBP is about 2, 3, 4, 5, 6, 7 8, 9, 10, 12, 15, 20, 25, 30, 35, 40, 45, or 50 amino acids in length. In an embodiment, the CBP is between 3 and 12 amino acids in length or 4 and 10 amino acids in length. The CBP amino acid sequence may be identical to the naturally occurring binding domain sequence or may be a conservatively substituted variant thereof. In an embodiment, the CAM ligand is a mammalian protein. In an embodiment, the CAM ligand is a human protein selected from the group of proteins listed in Table 1 below. In an embodiment, the CBP comprises a cell binding sequence listed in Table 1 below or a conservatively substituted variant thereof. In an embodiment, the CBP comprises at least one of the cell binding sequences listed in Table 1 below. In an embodiment, the CBP consists essentially of a cell binding sequence listed in Table 1 below. In an embodiment, the CBP is an RGD peptide, which means the peptide comprises the amino acid sequence RGD and optionally comprises one or more additional amino acids located at one or both of the N-terminus and C-terminus. In an embodiment, the CBP is a cyclic peptide comprising RGD, e.g., one of the cyclic RGD peptides described in Vilaca, H. et al., Tetrahedron 70 (35):5420-5427 (2014). In an embodiment, the CBP is a linear peptide comprising RGD and is less than 6 amino acids in length. In an embodiment, the CBP is a linear peptide that consists essentially of RGD or RGDSP.

TABLE 1
Exemplary CAM Ligand Proteins and Cell Binding
Sequences

| Protein | Cell Binding Sequence |
|---|---|
| E-cadherin | SWELYYPLRANL |
| N-cadherin | HAVDI |
| Collagen I | DGEA |
| Collagen IV | FYFDLR |
| | GFOGER |
| | P(GPP)₅GFOGER(GPP)₅ |
| | (wherein O is 4-hydroxyproline) |
| Elastin | VAPG |
| Fibrinogen | RGD |
| | GPR |
| Fibronectin | RGD |
| | KQAGDV |
| | PHSRN |
| | PHSRNGGGGGGRGDS |
| | REDV |
| Laminin | IKVAV |
| | SRARKQAASIKVAVADR |
| | LRE |
| | KQLREQ YIGSR |
| Nidogen-1 | RGD |
| Osteopontin | SVVYGLR |
| Tenascin C (TN-C) | AEIDGIEL |
| Tenascin-R | RGD |
| Tenascin-X | RGD |
| Thrombospondin | VTCG |
| | SVTCG |
| Vitronectin | RGD |
| Von Willebrand | RGD |
| Factor | |

“CBP-polymer”, as used herein, means a polymer comprising at least one CBP covalently attached to the polymer via a linker. In an embodiment, the polymer is not a peptide or a polypeptide. In an embodiment, the polymer in a CBP-polymer does not contain any amino acids. In an embodiment, the polymer in a CBP-polymer is a synthetic or naturally occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the C-terminus of an amino acid linker is joined to the N-terminus of the CBP and the N-terminus of the amino acid linker is joined to at least one pendant carboxyl group in the polysaccharide via an amide bond. In an embodiment, the structure of the linker-CBP is expressed as G₍₁₋₄₎-CBP, meaning that the linker has one, two, three or four glycine residues. In an embodiment, one or more of the monosaccharide moieties in a CBP-polysaccharide, e.g., a CBP-alginate) is not modified with the CBP, e.g, the unmodified moiety has a free carboxyl group or lacks a modifiable pendant carboxyl group. In an embodiment, the number of polysaccharide moieties with a covalently attached CBP is less than any of the following values: 99%, 95%, 90%, 80%, 70%, 60%, 50%, 40% 30%, 20%, 10%, 5%, 1%.

In an embodiment, the concentration of the linker-CBP modification in an CBP-polymer (e.g., a MMW alginate covalently modified with GRGDSP) is 0.05 to 2.0, 0.1 to 1.0, 0.2 to 0.8, 0.3 to 0.7, 0.3 to 0.6, 0.4 to 0.6 micromoles of the linker-CBP moiety per g of the CBP-polymer in solution (e.g., saline solution) with a viscosity of 80-120 cP, as determined by any assay that is capable of quantitating the amount of a peptide conjugated to a polymer, e.g., a quantitative peptide concentration assay described herein. Unless otherwise explicitly stated or readily apparent from the context, a specifically recited numerical concentration, concentration range, density or density range for a CBP in a CBP-polymer refers to the concentration of conjugated CBP molecules, i.e., it does not include any residual free (e.g., unconjugated) CBP that may be present in the CBP-polymer.

“CBP-density”, as used herein, refers to the amount or concentration of a linker-CBP moiety in a CBP-polymer, e.g., an alginate modified with G₁₋₃ RGD or G₁₋₃ RGDSP, unless otherwise explicitly stated herein.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 2 below.

TABLE 2
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface Exposure | Asp, Glu, Asn, Aln, His, Arg, Lys |
| | Cys, Ser, Tyr, Pro, Ala, Gly, Trp, Tyr |
| | Met, Ile, Leu, Val, Phe |
| Secondary Structure Propensity | Ala, Glu, Aln, His, Lys, Met, Leu, Arg |
| | Cys, Thr, Ile, Val, Phe, Tyr, Trp |
| | Ser, Gly, Pro, Asp, Asn |
| Evolutionary Conservation | Asp, Glu |
| | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, a polypeptide that consists essentially of a recited amino acid sequence may also include one or more amino acids, including substitutions in the recited amino acid sequence, of one or more amino acid residues, which do not materially affect the relevant biological activity of the cell-binding peptide or the therapeutic protein, respectively. As another non-limiting example, a polypeptide that consists essentially of a recited amino acid sequence may contain one or more covalently attached moieties (e.g., a radioactive or fluorescent label) that do not materially change the relevant biological activity of the cell-binding peptide, e.g., its ability to increase the viability or productivity of encapsulated cells as described herein.

“Derived from”, as used herein with respect to cells, refers to cells obtained from tissue, cell lines, or cells, which optionally are then cultured, passaged, differentiated, induced, etc. to produce the derived cells. For example, mesenchymal stem cells can be derived from mesenchymal tissue and then differentiated into a variety of cell types.

“Device”, as used herein, refers to any implantable object (e.g., a particle, a hydrogel capsule, an implant, a medical device), which contains cells (e.g., live cells) capable of expressing a therapeutic agent following implant of the device, and has a configuration that supports the viability of the cells by allowing cell nutrients to enter the device. In some embodiments, the device is a semi-permeable device, wherein it allows release from the device of metabolic byproducts and/or the therapeutic agent generated by the live cells.

“Effective amount”, as used herein, refers to an amount of a device, a device composition, or a component of the device or device composition, e.g, a plurality of hydrogel capsules comprising a cell, e.g., an engineered cell, or an agent, e.g., a therapeutic agent, produced by a cell sufficient to elicit a biological response, e.g., to treat a disease, disorder, or condition. In some embodiments, the term “effective amount” refers to the amount of a component of the device, e.g., number of cells in the device or the concentration of a polypeptide bound to a modified polymer within a polymer composition. As will be appreciated by those of ordinary skill in this art, the effective amount may vary depending on such factors as the desired biological endpoint, the pharmacokinetics of the therapeutic agent, composition or device (e.g., capsule, particle), the condition being treated, the mode of administration, and the age and health of the subject. An effective amount encompasses therapeutic and prophylactic treatment. As an example, an effective amount of a polypeptide (e.g., a CBP) may enhance the viability of the cells in the device (e.g., number of live cells) and/or increase the level of a therapeutic agent expressed and/or secreted by the cells. An effective amount of a device, composition or component, e.g., a CBP, may be determined by any technique known in the art or described herein.

“Engineered cell,” as used herein, is a cell having a non-naturally occurring alteration, and typically comprises a nucleic acid sequence (e.g., DNA or RNA) or a polypeptide not present (or present at a different level than) in an otherwise similar cell under similar conditions that is not engineered (an exogenous nucleic acid sequence). In an embodiment, an engineered cell comprises an exogenous nucleic acid (e.g., a vector or an altered chromosomal sequence). In an embodiment, an engineered cell comprises an exogenous polypeptide or an exogenous nucleic acid sequence, e.g., a sequence, e.g., DNA or RNA, not present in a similar cell that is not engineered. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, an engineered cell comprises a polypeptide present at a level or distribution which differs from the level found in a similar cell that has not been engineered. In an embodiment, an engineered cell comprises an RPE engineered to produce an RNA or a polypeptide. For example, an engineered cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, an engineered cell comprises an exogenous nucleic acid sequence that comprises a chromosomal or extra-chromosomal nucleic acid sequence comprising a sequence that encodes a polypeptide, or which is expressed as a polypeptide. In an embodiment, the polypeptide is encoded by a codon optimized sequence to achieve higher expression of the polypeptide than a naturally occurring coding sequence. The codon optimized sequence may be generated using a commercially available algorithm, e.g., GeneOptimizer (ThermoFisher Scientific), OptimumGene™ (GenScript, Piscataway, N.J. USA), GeneGPS® (ATUM, Newark, Calif. USA), or Java Codon Adaptation Tool (JCat, www.jcat.de, Grote, A. et al., Nucleic Acids Research, Vol 33, Issue suppl_2, pp. W526-W531 (2005). In an embodiment, an engineered cell (e.g., an RPE cell) comprises an exogenous nucleic acid sequence that modulates the conformation or expression of an endogenous sequence. In an embodiment, an engineered cell (e.g., RPE cell) is cultured from a population of stably transfected cells, or from a monoclonal cell line.

“Factor VII protein” or “FVII protein” as used herein, means a polypeptide that comprises the amino acid sequence of a naturally occurring factor VII protein or variant thereof that has a FVII biological activity, e.g., promoting blood clotting, as determined by an art-recognized assay, unless otherwise specified. Naturally occurring FVII exists as a single chain zymogen, a zymogen-like two-chain polypeptide and a fully activated two-chain form (FVIIa). In some embodiments, reference to FVII includes single-chain and two-chain forms thereof, including zymogen-like and FVIIa. FVII proteins that may be produced by a device described herein, e.g., a device containing engineered RPE cells, include wild-type primate (e.g., human), porcine, canine, and murine proteins, as well as variants of such wild-type proteins, including fragments, mutants, variants with one or more amino acid substitutions and/or deletions. In some embodiments, a variant FVII protein is capable of being activated to the fully activated two-chain form (Factor VIIa) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US published application No. US20080058255. Factor VII biological activity may be quantified by an art recognized assay, unless otherwise specified. For example, FVII biological activity in a sample of a biological fluid, e.g., plasma, may be quantified by (i) measuring the amount of Factor Xa produced in a system comprising tissue factor (TF) embedded in a lipid membrane and Factor X (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa).

“Factor VIII protein” or “FVIII protein” as used herein, means a polypeptide that comprises the amino acid sequence of a naturally occurring factor VIII polypeptide or variant thereof that has an FVIII biological activity, e.g., coagulation activity, as determined by an art-recognized assay, unless otherwise specified. FVIII proteins that may be expressed by a device described herein, e.g., a device containing engineered RPE cells, include wild-type primate (e.g., human), porcine, canine, and murine proteins, as well as variants of such wild-type proteins, including fragments, mutants, variants with one or more amino acid substitutions and/or deletions, B-domain deletion (BDD) variants, single chain variants and fusions of any of the foregoing wild-type or variants with a half-life extending polypeptide. In an embodiment, the cells are engineered to encode a precursor factor VIII polypeptide (e.g., with the signal sequence) with a full or partial deletion of the B domain. In an embodiment, the cells are engineered to encode a single chain factor VIII polypeptide which contains a variant FVIII protein preferably has at least 50%, 75%, 90% or more (including >100%) of the coagulation activity of the corresponding wild-type factor VIII. Assays for measuring the coagulation activity of FVIII proteins include the one stage or two stage coagulation assay (Rizza et al., 1982, Coagulation assay of FVIII:C and FIXa in Bloom ed. The Hemophelias. NY Churchill Livingston 1992) or the chromogenic substrate FVIII:C assay (Rosen, S. 1984. Scand J Haematol 33:139-145, suppl.).

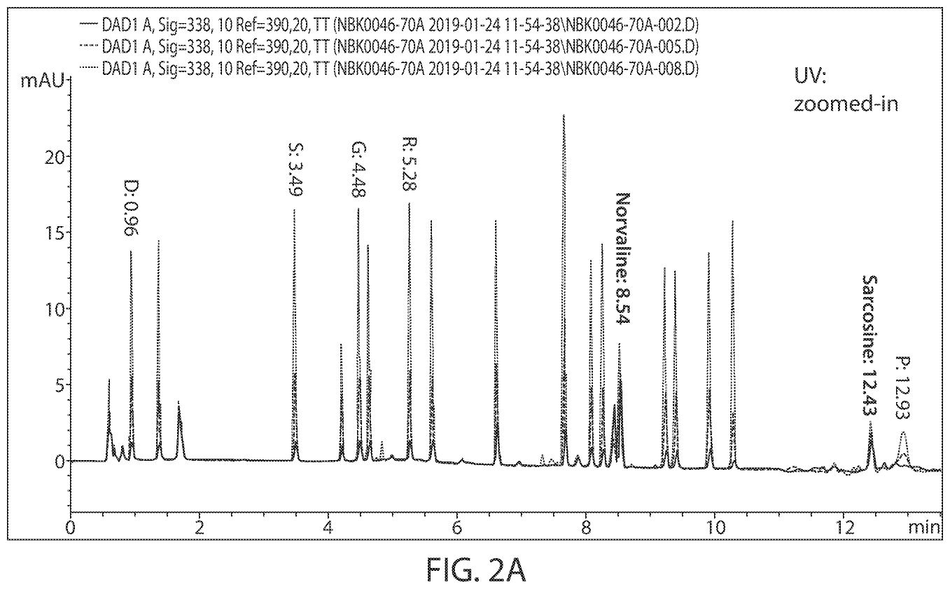

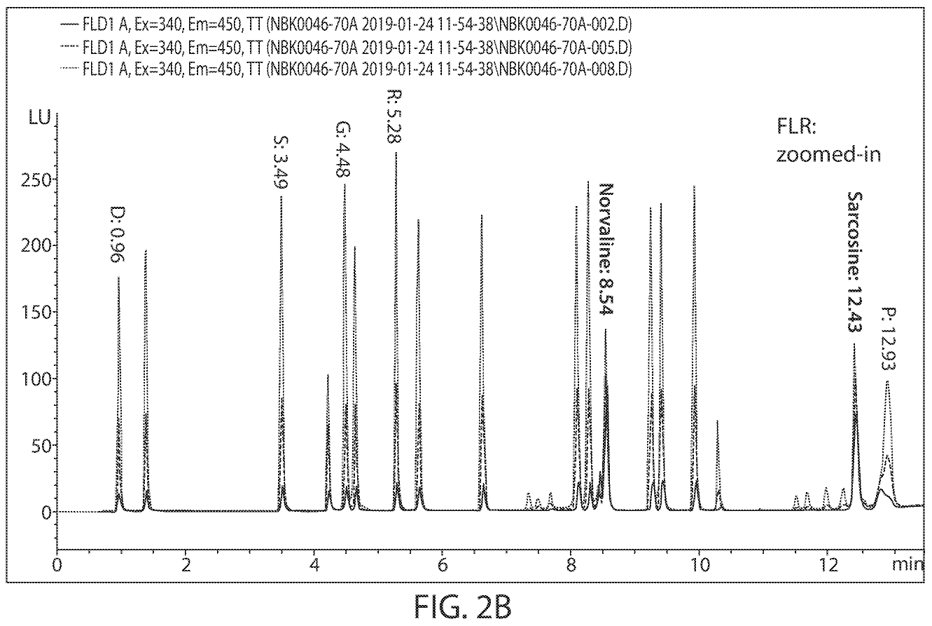

A number of FVIII-BDD variants are known, and include, e.g., variants with the full or partial B-domain deletions disclosed in any of the following U.S. Pat. Nos.: 4,868,112 (e.g., col. 2, line 2 to col. 19, line 21 and table 2); 5,112,950 (e.g., col. 2, lines 55-68, FIG. 2, and example 1); 5,171,844 (e.g., col. 4, line 22 to col. 5, line 36); 5,543,502 (e.g., col. 2, lines 17-46); U.S. Pat. Nos. 5,595,886; 5,610,278; 5,789,203 (e.g., col. 2, lines 26-51 and examples 5-8); 5,972,885 (e.g., col. 1, lines 25 to col. 2, line 40); 6,048,720 (e.g., col. 6, lines 1-22 and example 1); U.S. Pat. Nos. 6,060,447; 6,228,620; 6,316,226 (e.g., col. 4, line 4 to col. 5, line 28 and examples 1-5); 6,346,513; 6,458,563 (e.g., col. 4, lines 25-53) and 7,041,635 (e.g., col. 2, line 1 to col. 3, line 19, col. 3, line 40 to col. 4, line 67, col. 7, line 43 to col. 8, line 26, and col. 11, line 5 to col. 13, line 39). In some embodiments, a FVIII-BDD protein produced by a device described herein (e.g., expressed by engineered cells contained in the device) has one or more of the following deletions of amino acids in the B-domain: (i) most of the B domain except for amino-terminal B-domain sequences essential for intracellular processing of the primary translation product into two polypeptide chains (WO 91/09122); (ii) a deletion of amino acids 747-1638 (Hoeben R. C., et al. J. Biol. Chem. 265 (13): 7318-7323 (1990)); amino acids 771-1666 or amino acids 868-1562 (Meulien P., et al. Protein Eng. 2(4):301-6 (1988); amino acids 982-1562 or 760-1639 (Toole et al., Proc. Natl. Acad. Sci. U.S.A. 83:5939-5942 (1986)); amino acids 797-1562 (Eaton et al., Biochemistry 25:8343-8347 (1986)); 741-1646 (Kaufman, WO 87/04187)), 747-1560 (Sarver et al., DNA 6:553-564 (1987)); amino acids 741-1648 (Pasek, WO 88/00831)), amino acids 816-1598 or 741-1689 (Lagner (Behring Inst. Mitt. (1988) No 82:16-25, EP 295597); a deletion that includes one or more residues in a furin protease recognition sequence, e.g., LKRHQR (SEQ ID NO: 65) at amino acids 1643-1648, including any of the specific deletions recited in U.S. Pat. No. 9,956,269 at col. 10, line 65 to col. 11, line 36.

In other embodiments, a FVIII-BDD protein retains any of the following B-domain amino acids or amino acid sequences: (i) one or more N-linked glycosylation sites in the B-domain, e.g., residues 757, 784, 828, 900, 963, or optionally 943, first 226 amino acids or first 163 amino acids (Miao, H. Z., et al., Blood 103(a): 3412-3419 (2004), Kasuda, A., et al., J. Thromb. Haemost. 6: 1352-1359 (2008), and Pipe, S. W., et al., J. Thromb. Haemost. 9: 2235-2242 (2011).

In some embodiments, the FVIII-BDD protein is a single-chain variant generated by substitution of one or more amino acids in the furin protease recognition sequence (LKRHQR (SEQ ID NO: 65) at amino acids 1643-1648) that prevents proteolytic cleavage at this site, including any of the substitutions at the R1645 and/or R1648 positions described in U.S. Pat. Nos. 10,023,628, 9,394,353 and 9,670,267.

In some embodiments, any of the above FVIII-BDD proteins may further comprise one or more of the following variations: a F3095 substitution to improve expression of the FVIII-BDD protein (Miao, H. Z., et al., Blood 103(a): 3412-3419 (2004); albumin fusions (WO 2011/020866); and Fc fusions (WO 04/101740).

All FVIII-BDD amino acid positions referenced herein refer to the positions in full-length human FVIII, unless otherwise specified.

“Factor IX protein” or “FIX protein”, as used herein, means a polypeptide that comprises the amino acid sequence of a naturally occurring factor IX protein or variant thereof that has a FIX biological activity, e.g., coagulation activity, as determined by an art-recognized assay, unless otherwise specified. FIX is produced as an inactive zymogen, which is converted to an active form by factor XIa excision of the activation peptide to produce a heavy chain and a light chain held together by one or more disulfide bonds. FIX proteins that may be produced by devices described herein (e.g., a semi-permeable device containing engineered RPE cells) include wild-type primate (e.g., human), porcine, canine, and murine proteins, as well as variants of such wild-type proteins, including fragments, mutants, variants with one or more amino acid substitutions and/or deletions and fusions of any of the foregoing wild-type or variant proteins with a half-life extending polypeptide. In an embodiment, cells are engineered to encode a full-length wild-type human factor IX polypeptide (e.g., with the signal sequence) or a functional variant thereof. A variant FIX protein preferably has at least 50%, 75%, 90% or more (including >100%) of the coagulation activity of wild-type factor VIX. Assays for measuring the coagulation activity of FIX proteins include the Biophen Factor IX assay (Hyphen BioMed) and the one stage clotting assay (activated partial thromboplastin time (aPTT), e.g., as described in EP 2 032 607, thrombin generation time assay (TGA) and rotational thromboelastometry, e.g., as described in WO 2012/006624.

A number of functional FIX variants are known and may be expressed by engineered cells encapsulated in a device described herein, including any of the functional FIX variants described in the following international patent publications: WO 02/040544 at page 4, lines 9-30 and page 15, lines 6-31; WO 03/020764 in Tables 2 and 3 at pages 14-24, and at page 12, lines 1-27; WO 2007/149406 at page 4, line 1 to page 19, line 11; WO 2007/149406 A2 at page 19, line 12 to page 20, line 9; WO 08/118507 at page 5, line 14 to page 6, line 5; WO 09/051717 at page 9, line 11 to page 20, line 2; WO 09/137254 at page 2, paragraph [006] to page 5, paragraph [011] and page 16, paragraph [044] to page 24, paragraph [057]; WO 09/130198 A2 at page 4, line 26 to page 12, line 6; WO 09/140015 at page 11, paragraph [0043] to page 13, paragraph [0053]; WO 2012/006624; WO 2015/086406.

In certain embodiments, the FIX polypeptide comprises a wild-type or variant sequence fused to a heterologous polypeptide or non-polypeptide moiety extending the half-life of the FIX protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof. An exemplary FIX polypeptide is the rFIXFc protein described in WO 2012/006624, which is an FIXFc single chain (FIXFc-sc) and an Fc single chain (Fc-sc) bound together through two disulfide bonds in the hinge region of Fc.

FIX variants also include gain and loss of function variants. An example of a gain of function variant is the “Padua” variant of human FIX, which has a L (leucine) at position 338 of the mature protein instead of an R (arginine) (corresponding to amino acid position 384 of SEQ ID NO:2), and has greater catalytic and coagulant activity compared to wild-type human FIX (Chang et al., J. Biol. Chem., 273:12089-94 (1998)). An example of a loss of function variant is an alanine substituted for lysine in the fifth amino acid position from the beginning of the mature protein, which results in a protein with reduced binding to collagen IV (e.g., loss of function). “Interleukin-2 protein” or “IL-2 protein”, as used herein means a polypeptide comprising the amino acid sequence of a naturally-occurring IL-2 protein or variant thereof that has an IL-2 biological activity, e.g., activate IL-2 receptor signaling in Treg cells, as determined by an art-recognized assay, unless otherwise specified. IL-2 proteins that may be produced by a device described herein, e.g., a device containing engineered RPE cells, include wild-type primate (e.g., human), porcine, canine, and murine proteins, as well as variants of such wild-type proteins. A variant IL-2 protein preferably has at least 50%, 75%, 90% or more (including >100%) of the biological activity of the corresponding wild-type IL-2. Biological activity assays for IL-2 proteins are described in U.S. Pat. No. 10,035,836, and include, e.g., measuring the levels of phosphorylated STATS protein in Treg cells compared to CD4+CD25−/low T cells or NK cells. Variant IL-2 proteins that may be produced by a device of the present disclosure (e.g., a device containing engineered RPE cells) include proteins with one or more of the following amino acid substitutions: N88R, N88I, N88G, D20H, Q126L, Q126F, and C125S or C125A.

“Medium molecular weight alginate,” or “MMW-Alg” as used herein means an alginate with an approximate molecular weight of 75 kDa to 150 kDa.

“Polymer composition”, as used herein, is a composition (e.g., a solution, mixture) comprising one or more polymers. As a class, “polymers’ includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, refers to a polymer comprising amino acid residues linked through peptide bonds and having at least 2, and in some embodiments, at least 3, 4, 5, 6, 7, 8, 9, 10, 12, 15, 20, 25, 30, 35, 40, 45, 50, 75, 100, 150 or 200 amino acid residues.

“Prevention,” “prevent,” and “preventing” as used herein refers to a treatment that comprises administering or applying a therapy, e.g., administering a composition of devices encapsulating cells (e.g., as described herein), prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

“Reference device”, as used herein with respect to a claimed device (e.g., hydrogel capsule), means a device (e.g., hydrogel capsule) that: (i) lacks a particular feature, e.g., a CBP, (ii) encapsulates in the cell-containing compartment about the same quantity of cells of the same cell type(s) as in the claimed device, and (iii) has a substantially similar polymer composition and structure as in the claimed device other than lacking the particular feature (e.g., the CBP). In an embodiment, the number of live cells in the cell-containing compartment of a reference device is within 80% to 120%, or within 90% to 110%, of the number of live cells in the cell-containing compartment of the claimed device. In an embodiment, the cells in the reference and claimed devices are obtained from the same cell culture. In an embodiment, a substantially similar polymer composition means all polymers in the reference and claimed device, including the polymer component of any CBP-polymer, as applicable, are of the same chemical and molecular weight class (e.g., an alginate with high G content and the same molecular weight range). For example, in an embodiment, the cell-containing compartment of a CBP-null reference device is formed from the unmodified version of the polymer (e.g., alginate) in the CBP-polymer used to form the cell-containing compartment of the claimed device. In an embodiment, a substantially similar structure means the reference and claimed devices have the same number of compartments (e.g., one, two, three, etc.) and about the same size and shape.

“Refractive index” as used herein refers to a dimensionless value representing the relative ratio of the speed of light in a medium (e.g., a sample) relative to the speed of light in a vacuum. The refractive index may be used to describe the phase velocity of light through the medium, e.g., how much the path of light is refracted when entering the medium (e.g., the sample). The refractive index of a sample may be measured by a refractometer and is temperature and wavelength dependent. Exemplary refractive indices include water (nD=1.333 at 20° C.) and ethanol (nD=1.36 at 20° C.).

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455. In an embodiment, the specified percentage of identical nucleotide or amino acid positions is at least about 80%, 85%, 90%, 91%, 92%, 93%, 94%, 95%, 96%, 97%, 98%, 99% or higher.

“Subject” as used herein refers to a human or non-human animal. In an embodiment, the subject is a human (i.e., a male or female), e.g., of any age group, a pediatric subject (e.g., infant, child, adolescent) or adult subject (e.g., young adult, middle-aged adult, or senior adult). In an embodiment, the subject is a non-human animal, for example, a mammal (e.g., a mouse, a dog, a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Features of Polymer Compositions

The present disclosure features methods of evaluating polymer compositions comprising at least one polymer modified with a polypeptide. For example, using the methods described herein, the total concentration of polypeptide bound to the modified polymer(s) may be determined. The polymer compositions may be used to encapsulate cells (e.g., live cells) expressing a therapeutic agent, forming a semi-permeable device capable of releasing the therapeutic agent upon implant of the device into a subject, e.g., a human or other mammalian subject. The methods described herein may provide a means for reliably determining the concentration of a polypeptide of a modified polymer within the polymer composition, which may be useful, e.g., to gauge the stability of polypeptide modification over time.

The polypeptide bound to a modified polymer described herein may comprise a naturally occurring peptide sequence or non-naturally occurring peptide sequence, as well as combinations, fragments or variants thereof. In an embodiment, the polypeptide comprises a sequence derived from a hormone, enzyme, cell-binding peptide, antibody, cytokine (e.g., a pro-inflammatory cytokine or an anti-inflammatory cytokine), growth factor, clotting factor, or lipoprotein sequence, or a fragment or variant thereof. The polypeptide may comprise naturally occurring amino acids, non-naturally occurring amino acids, or a combination thereof. In addition, a polypeptide for use with the present disclosure may be modified in some way, e.g., via chemical or enzymatic modification (e.g., glycosylation, phosphorylation). In some embodiments, the polypeptide has about 2, 3, 4, 5, 6, 7, 8, 9, 10, 12, 14, 16, 18, 20, 25, 30, 35, 40, 45, or less than 50 amino acids. In some embodiment, the polypeptide has more than about 2, 3, 4, 5, 6, 7, 8, 9, 10, 12, 14, 16, 18, 20, 25, 30, 35, 40, 45 amino acids. In some embodiments, the polypeptide is between 2-100 amino acids in length, e.g., between 2-75 amino acids, 2-50 amino acids, 2-40 amino acids, 2-30 amino acids, 2-25 amino acids, 2-20 amino acids, 2-15 amino acids, 2-10 amino acids, 2-8 amino acids, 2-6 amino acids, or 2-4 amino acids in length.

In some embodiments, the polypeptide bound to a modified polymer has an average molecular weight of 1 kD, 2 kD, 2.5 kD, 5 kD, 10 kD, 25 kD, 50 kD, 100 kD, 150 kD, 200 kD, 250 kD, 500 kD, or more. In some embodiments, the polypeptide has an average molecular weight less than 500 kD, 250 kD, 200 kD, 150 kD, 100 kD, 50 kD, 25 kD, 10 kD, 5 kD, 2.5 kD, 2 kD, 1 kD, or less. In some embodiments, polypeptide has an average molecular weight of more than 5 kD. In some embodiments, the peptide or polypeptide has an average molecular weight of more than 10 kD. In some embodiments, the peptide or polypeptide has an average molecular weight between 1-500 kD, e.g., between 1-250 kD, 1-100 kD, 1-50 kD, 1-25 kD, 1-10 kD, 1-5 kD, 1-2.5, kD, 5-500 kD, 5-250 kD, 5-100 kD, 5-50 kD, 5-10 kD, 10-500 kD, 10-250 kD, 10-100 kD, 10-50 kD, 50-500 kD, 50-250 kD, 50-100 kD, 100-500 kD, or 100-250 kD.

In some embodiments, the polypeptide bound to a modified polymer comprises is a cell-binding polypeptide (CBP), as described herein. In some embodiments, the CBP comprises a sequence such as RGD, RGDSP, DGEA, PHSRN, YIGSR, a peptide comprising or consisting essentially of any of the cell binding sequences listed in Table 1 herein, or a mixture of 2, 3 or more of these CBPs (e.g., RGD+DGEA, RGD+PHSRN, RGD+DGEA+PHSRN).

Each polypeptide (e.g., a CBP) may be is attached to a polymer in the polymer composition via a linker. The linker may be an amino acid linker and may be, for example, between 1 and 20 amino acids in length. In an embodiment, the amino acid linker comprises a glycine residue. In an embodiment, the amino acid linker comprises a plurality of glycine residues, e.g., more than two glycine residues or more than three glycine residues. In an embodiment, the amino acid linker consists essentially of one, two or three glycine residues. In an embodiment, the polypeptide is a CBP and the amino acid linker comprises a single glycine residue, e.g., attached to the N-terminus of the CBP. In an embodiment, the polypeptide is GRGD or GRGDSP.

In an embodiment, the polymer composition comprises a single polymer modified with a polypeptide. In another embodiment, the polymer composition comprises a plurality of polymers modified with a polypeptide, e.g., at least 2 modified polymers, at least 3 modified polymers, or at least 4 modified polymers. The polymer compositions may comprise polymers modified with polypeptides having the same sequence, or polymers modified with polypeptides having different sequences. The polymer compositions may comprise a mixture of modified polymers and unmodified polymers.

In an embodiment, the polymer composition comprises a first modified polymer, e.g., modified with RGD, RGDSP, DGEA, PHSRN, YIGSR, or a polypeptide comprising or consisting essentially of any of the cell binding sequences listed in Table 1 herein. In another embodiment, the polymer composition comprises a second modified polymer, e.g., modified with RGD, RGDSP, DGEA, PHSRN, YIGSR, or a polypeptide comprising or consisting essentially of any of the cell binding sequences listed in Table 1 herein.

Each polypeptide may further comprise a terminal capping group or protecting group on the free terminus of the polypeptide, for example, the terminus of the polypeptide not covalently bound to a polymer of the polymer composition. A terminal capping group or protecting group may prevent the terminal amino acid residue from unwanted or unnecessary reactions and/or reduce or prevent degradation or modification of the polypeptide. Exemplary capping groups include alkyl groups, ethers, amides, and the like. In an embodiment, the linker-CBP comprises GRGD or GRGDSP and a terminal capping group. In an embodiment, the linker-CBP comprises GRGD or GRGDSP and does not comprise a terminal capping group.

Each of the modified and unmodified polymers in the polymer composition may be a linear, branched, or cross-linked polymer, or a polymer of selected molecular weight ranges, degree of polymerization, viscosity or melt flow rate. Exemplary polymers within the polymer composition include naturally occurring polymers and non-naturally occurring polymers (e.g., synthetic polymers). For example, the polymer composition may include polystyrene, polyethylene, polypropylene, polyacetylene, poly(vinyl chloride) (PVC), polyvinyl alcohol (PVA), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), racemic poly(lactic acid), polycarbonates, (e.g., polyamides (e.g., nylon)), fluoroplastics, carbon fiber, agarose, alginate, chitosan, and blends or copolymers thereof. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymer. The amount of a modified polymer in the polymer composition (e.g., by % weight of the polymer composition, actual weight of the polymer) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In an embodiment, the polymer composition may comprise a polymer selected from alginate, chitosan, hyaluronate, a polyethylene glycol (PEG), a polyacrylamide, gelatin, poly(L-lactic acid) (PLLA), poly(lactic glycolic acid) (PLGA), carboxymethylcellulose, and carboxymethylchitosan. In an embodiment, the polymer composition does not comprise a polyamide polymer, e.g, other than the polypeptide bound to the modified polymer. In addition to the polymers described herein, the polymer composition in polymer composition may contain one or more unmodified naturally-occurring or synthetic polymers of any of the above-recited polymer classes to help provide structural integrity to the semi-permeable device and/or help provide a scaffold for supporting the encapsulated cells.

In an embodiment, the modified polymer is an alginate. Alginate is a polysaccharide made up of β-D-mannuronic acid (M) and α-L-guluronic acid (G). In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the ratio of G:M is at least 1.3, 1.5 or greater than 1.5. In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In an embodiment, the polymer composition comprises a modified alginate and an unmodified alginate.

Polymers may be modified with a polypeptide using any of a variety of methods known in the art, see, e.g., Jeon O, et al., Biodegradable, photocrosslinked alginate hydrogels with independently tailorable physical properties and cell adhesivity. Tissue Eng. Part A 16:2915-2925 (2010); Rowley, J. A. et al., Biomaterials 20:45-53 (1999). For example, when the polymer to be modified is an alginate, the N-terminus of a polypeptide that consists essentially of an amino acid linker sequence (of one to several amino acids) and a cell binding amino acid sequence can be covalently attached to the alginate using an approach similar to that described in Example 1 herein.

In some embodiments, the polymer composition further comprises a polymer chemically modified with a small molecule, e.g., that has afibrotic properties (i.e., an afibrotic polymer). For example, the afibrotic polymer may be an afibrotic alginate. The alginate in the afibrotic polymer may be the same or different than any unmodified alginate that is present in the polymer composition. Exemplary small molecules and related modified polymers include those disclosed in any one of International Patent Publications WO 2012/167223, WO 2016/019391, WO 2017/075630, WO 2017/075631, WO 2018/067615, and WO2019/169333, each of which is incorporated herein by reference in its entirety.

The modified polymers described herein may be used to prepare a semi-permeable device encapsulating cells. In an embodiment, the semi-permeable devices may comprise a single type of modified polymer, or a plurality of types of modified polymers. These semi-permeable devices comprise at least one cell-containing compartment that comprises a plurality of cells (e.g., live cells). In an embodiment, the device contains two, three, four or more cell-containing compartments. The cells may be a variety of different cell types (e.g., human cells), including epithelial cells, endothelial cells, fibroblast cells, mesenchymal stem cells, keratinocyte cells and islet cells. Exemplary cell types include the cell types recited in WO 2017/075631, which is incorporated herein by reference in its entirety. In an embodiment, the cells are engineered cells, e.g., engineered to express a therapeutic agent (e.g., a protein, e.g., an antibody, enzyme, blood clotting factor, hormone, or growth factor).

In an embodiment, cells contained in a device of the disclosure comprise RPE cells or MSC cells (e.g., engineered RPE cells or engineered MSC cells). In some embodiments, the semi-permeable device does not comprise an islet cell. In an embodiment, the cells contained in a device of the disclosure have one or more of the following characteristics: (i) are not capable of producing insulin (e.g., insulin A-chain, insulin B-chain, or proinsulin) in an amount effective to treat diabetes or another disease or condition that may be treated with insulin; (ii) not capable of producing insulin in a glucose-responsive manner; or (iii) not derived from an induced pluripotent stem cell that was engineered or differentiated into insulin-producing pancreatic beta cells.

In an embodiment, the plurality of cells is in the form of a cell suspension prior to being encapsulated within a semi-permeable device described herein, e.g., a hydrogel capsule. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In some embodiments, the cells in the plurality are engineered to produce a therapeutic agent. In an embodiment, the therapeutic agent is for the prevention or treatment of a disease, disorder, or condition, e.g., those described in WO 2017/075631. The therapeutic agent may be any biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule. Exemplary therapeutic agents include the agents listed in WO 2017/075631.

In some embodiments, the therapeutic agent is a protein, such as a hormone, enzyme, cytokine (e.g., a pro-inflammatory cytokine or an anti-inflammatory cytokine), growth factor, clotting factor, or lipoprotein. A peptide or polypeptide (e.g., a protein, e.g., a hormone, growth factor, clotting factor or coagulation factor, antibody molecule, enzyme, cytokine, cytokine receptor, or a chimeric protein including cytokines or a cytokine receptor) produced by an engineered cell can have a naturally occurring amino acid sequence, or may contain a variant of the naturally occurring sequence. The variant can be a naturally occurring or non-naturally occurring amino acid substitution, mutation, deletion or addition relative to the reference sequence, e.g., a naturally occurring sequence. The naturally occurring amino acid sequence may be a polymorphic variant. The naturally occurring amino acid sequence can be a human or a non-human amino acid sequence. In some embodiments, the naturally occurring amino acid sequence or naturally occurring variant thereof is a human sequence. In addition, a protein for use with the present disclosure may be modified in some way, e.g., via chemical or enzymatic modification (e.g., glycosylation, phosphorylation). In some embodiments, the protein has an average molecular weight of 5 kD, 10 kD, 25 kD, 50 kD, 100 kD, 150 kD, 200 kD, 250 kD, 500 kD, or more.

In some embodiments, the protein is a clotting factor or a coagulation factor, e.g., a blood clotting factor or a blood coagulation factor. In some embodiments, the protein is a protein involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor (vWF), prekallikrein, heparin cofactor II, high molecular weight kininogen (e.g., Fitzgerald factor), antithrombin III, and fibronectin. In some embodiments, the protein is an anti-clotting factor, such as Protein C.

In some embodiments, the protein is a replacement therapy or a replacement protein. In some embodiments, the replacement therapy or replacement protein is a clotting factor or a coagulation factor, e.g., Factor VIII (e.g., comprises a naturally occurring human Factor VIII amino acid sequence or a variant thereof) or Factor IX (e.g., comprises a naturally occurring human Factor IX amino acid sequence or a variant thereof).

In some embodiments, the encapsulated cells described herein are engineered to express a human Factor VIII protein, e.g., a recombinant Factor VIII. In some embodiments, the recombinant Factor VIII is a B-domain-deleted recombinant Factor VIII (FVIII-BDD). In some embodiments, the cells are engineered to express a Factor IX, e.g., a human Factor IX (FIX) protein.

In some embodiments, the encapsulated cells are derived from a human RPE cell line and comprise an exogenous nucleic acid sequence which comprises a promoter sequence operably linked to a coding sequence for a polypeptide. In an embodiment, the coding sequence is a codon-optimized FVIII-BDD coding sequence or a codon-optimized FIX-padua coding sequence.

In an embodiment, the concentration of polypeptide (e.g., CBP) present in the modified polymer is in an amount effective to increase viability of the cells and/or increase productivity of the cells at a timepoint after the semi-permeable device is implanted into an immune-compromised or immune-competent animal, e.g., immune-competent mice (e.g., the C57BL/6J mouse strain available from the Jackson Laboratory, Bar Harbor, Me. USA) as compared to a reference device that does not comprise a modified polymer comprising a polypeptide (e.g., a CBP). In an embodiment, the increase in cell viability and/or productivity is detectable at a desired timepoint after implant, e.g., at one or more of 1 day, 3 days, 5 days, 1 week, 2 weeks, 4 weeks, 8 weeks, 12 weeks, 24 weeks, 36 weeks and 48 weeks. In an embodiment, the effective amount of the polypeptide (e.g., the CBP) results in an increase in one or both of (i) cell viability by at least 10%, 25%, 50% or 100% when measured at 1 week, 2 weeks, 4 weeks or 12 weeks after implant and (ii) increases cell productivity by at least 1.25-fold, 1.5-fold, 2-fold, 5-fold, 8-fold or 10-fold when measured at 1 week, 2 weeks, 4 weeks or 12 weeks after implant. In an embodiment, the effective amount of the polypeptide (e.g., CBP) in the cell-containing compartment falls within a range between the minimally effective amount and a higher amount at which the cell viability and/or productivity are reduced compared to a reference device not comprising the polypeptide (e.g., the CBP), or compared to the a device containing a maximally-effective amount, e.g., the optimal amount, of the CBP in the cell-containing compartment. In an embodiment, the effective amount of a polypeptide (e.g., CBP) to achieve improved productivity after implant is determined for the desired combination of cells, peptide or polypeptide, therapeutic protein expressed by the cells, e.g., engineered ARPE-19 cells, specific protein (e.g., Factor VIII, Factor IX, Factor VII) and optionally including the particular coding sequence for the therapeutic protein.

In some embodiments, the concentration of polypeptide (e.g., a CBP) present in the polymer composition is correlated with a specific parameter, e.g., retention time on a chromatogram or area under a curve on a chromatogram. In an embodiment, the polypeptide is conjugated to the modified polymer or may be freely present in a sample, e.g., after hydrolysis. In an embodiment, the concentration of the polypeptide (e.g., conjugated to a polymer, e.g., alginate) is between 0.1% and 10%, (w/w). For example, the concentration of the polypeptide (e.g., a CBP, e.g., conjugated to a polymer, e.g., alginate) is between 0.5% and 10%, 0.5% and 5%, 0.5% and 3%, 1% and 5%, 1% and 4%, or 2% and 5% (w/w). In an embodiment, the concentration of the polypeptide (e.g., a CBP, e.g., conjugated to a polymer, e.g., alginate) is between 3% and 6% (w/w). In an embodiment, the concentration of the polypeptide (e.g., a CBP, e.g., conjugated to a polymer, e.g., alginate) is between 1% and 3% (w/w).

In some embodiments, the concentration of polypeptide (e.g., a CBP) present in the modified polymer is described as a ratio of particular amino acid components. In other embodiments, the identification of polypeptide (e.g., CBP) present in the modified polymer is described as a ration of particular amino acid components. For example, the ratio of a polypeptide (e.g., a CBP) may be described as a ratio of AA₁:AA₂:AA₃, etc, wherein AA₁, AA₂, and AA₃, etc refer to a first, second, third, and optional subsequent amino acids. In an embodiment, the CBP comprises RGD, RGDSP, DGEA, PHSRN, YIGSR, or a polypeptide comprising or consisting essentially of any of the cell binding sequences listed in Table 1 herein. In an embodiment, the ratio of amino acid components of the CBP is 1:1:1 (e.g., for RGD), 1:1:1:1 (e.g., for DGEA), 1:1:1:1:1 (e.g., for RGDSP, PHSRN, or YIGSR). The polypeptide may further comprise a linker, e.g., a linker-CBP. In an embodiment, the linker-CBP is GRGDSP and the ratio of amino acid components is 2:1:1:1:1 for G:R:D:S:P.

In some embodiments, the semi-permeable device is not any capsule, device, implant or other object disclosed in any of WO2012/112982, WO2012/167223, WO2014/153126, WO2016/019391, WO2016/187225, US2012-0213708, US 2016-0030359, and US 2016-0030360.

### Methods of Evaluating Modified Polymers in Compositions

The present disclosure features methods for evaluating a modified polymer comprising a polypeptide within a polymer composition. Evaluating may include determining the overall concentration of a modified polymer in a sample, determining the concentration of a polypeptide bound to a modified polymer in a sample, identifying the sequence of a polypeptide bound to a modified polymer in a sample, or querying a polymer composition for certain impurities. The methods described herein include subjecting a sample of a modified polymer to reaction conditions capable of either (i) releasing the polypeptide from the modified polymer and/or (ii) hydrolyzing the polypeptide into component amino acids. Once the polypeptide is liberated from the modified polymer and broken down into its component amino acids, the concentration of each component amino acids may be determined, e.g., through comparison with a set of standards. Finally, the concentration of the intact peptide or polypeptide bound to the modified polymer may be determined based on the concentration of each component amino acid.

Any reaction condition may be used in order to release the polypeptide from the modified polymer. For example, the polymer composition may be subjected to an acidic solution, a basic solution, an enzymatic solution, heat, light, microwave irradiation, hydrogenation, or a combination thereof. In an embodiment, the polymer composition is subjected to an acidic solution, such as solution comprising HCl, HBr, HF, H₂SO₄, HNO₃, HClO₄, CF₃COOH, CH₃COOH, or CF₃SO₃H, in order to release the polypeptide from the modified polymer. In an embodiment, an acidic solution has a pH less than 7, e.g., less 6.5, 6, 5.5, 5, 4.5, 4, 3.5, 3, 2.5, 2, 1.5, 1, or lower. The polymer composition may be subjected to heat, e.g., at a temperature greater than about 25° C., 40° C., 60° C., 80° C., 100° C., 120° C., or higher. In an embodiment, in order to release the polypeptide from the modified polymer, the polymer composition may be subjected to an acidic solution and heat, e.g., simultaneously. In an embodiment, the polymer composition is stirred while applying heat, e.g., using a stir plate.

In an embodiment, the polymer composition is subjected to microwave irradiation, e.g., for at least 1 seconds (s), 2 s, 5 s, 10 s, 15 s, 20 s, 25 s, 30 s, 35 s, 40 s, 45 s, 50 s, or 55 s, 1 minute (min), 2.5 min, 5 min, 10 min, 15 min, 20 min, 25 min, 30 min, 35 min, 40 min, 45 min, 50 min, 55 min, or 1 hour (hr). In an embodiment, the polymer composition is subject to microwave irradiation for between 1 min and 1 hr, e.g., between 1 min and 45 mins, 1 min and 30 mins, 1 min and 15 mins, 5 mins and 1 hr, 5 mins and 45 mins, 5 mins and 30 mins, 5 mins and 15 mins, 10 mins and 1 hr, 10 mins and 45 mins, 10 mins and 30 mins, 15 mins and 45 mins, and 15 mins and 30 mins. In an embodiment, the polymer composition is subjected to microwave irradiation for between 5 mins and 45 mins. In an embodiment, the polymer composition is stirred while applying microwave irradiation, e.g., at 100 revolutions per minute (rpm), 200 rpm, 300 rpm, 400 rpm, 500 rpm, 600 rpm, 700 rpm, 800 rpm or faster, e.g., in a microwave reactor (e.g., Anton-Paar Monowave microwave reactor). In an embodiment, the polymer composition is subjected to an acidic solution and microwave irradiation, e.g. simultaneously.

Similarly, any reaction condition may be used in order to hydrolyze the polypeptide into component amino acids. For example, the polymer composition may be subjected to an acidic solution, a basic solution, an enzymatic solution, heat, light, microwave irradiation, hydrogenation, or a combination thereof. In an embodiment, the polymer composition is subjected to an acidic solution, such as solution comprising HCl, HBr, HF, H₂SO₄, HNO₃, HClO₄, CF₃COOH, CH₃COOH, or CF₃SO₃H, in order to hydrolyze the polypeptide into component amino acids. In an embodiment, an acidic solution has a pH less than 7, e.g., less 6.5, 6, 5.5, 5, 4.5, 4, 3.5, 3, 2.5, 2, 1.5, 1, or lower. The polymer composition may be subjected to heat, e.g., at a temperature greater than about 25° C., 40° C., 60° C., 80° C., 100° C., 120° C., or higher. In an embodiment, in order to hydrolyze the polypeptide into component amino acids, the polymer composition may be subjected to an acidic solution and heat simultaneously.

In an embodiment, the release of the polypeptide from the modified polymer and the hydrolysis of the polypeptide into component amino acids are achieved in the same step. In an embodiment, the release of the r polypeptide from the modified polymer and the hydrolysis of the polypeptide into component amino acids are achieved in sequential steps or under different conditions.

The methods described herein may further comprise acquiring the concentration of each component amino acid of the polypeptide. To this end, in an embodiment, the composition of individual amino acids is subjected to a separation step. Any known separation step may be used to separate the individual amino acids from one another, e.g., to determine the concentration of each, such as filtration, electrophoresis, or chromatography. Exemplary chromatographic methods include size-exclusion chromatography, ion-exchange chromatography, gel filtration chromatography, reversed-phase chromatography, hydrophobic interaction chromatography, or thin layer chromatography. In an embodiment, reversed-phase chromatography is utilized to separate the individual amino acids in the composition.

In instances in which the polypeptide sequences described herein to do not contain a detectable moiety, the individual amino acids may be derivatized with a detection agent before or after separation. Derivatization may be used to improve the chromatographic behavior of the individual amino acids by, for example, making the amino acids more volatile, less reactive, or visible by a detection method. Exemplary detection agents include fluorophores, silylation, radioisotopes, antibodies or antibody fragments, enzymes, dyes, or other similar agents, as well as combinations thereof. Individual amino acids may be derivatized by reaction of the detection agent with the amino acid N-terminus, C-terminus, or side chain. In an embodiment, the detection agent comprises ninhydrin, ortho phthaladehyde, dansyl chloride, 9-fluorenylmethoxy chloroformate, or a combination thereof. In an embodiment, derivatization of the individual amino acids occurs prior to separation. In an embodiment, derivatization of the individual amino acids occurs after to separation. In an embodiment, derivatization of the individual amino acids occurs concurrent with separation.

In some embodiments, the individual amino acids may be derivatized through the use of a commercially available amino acid derivatization kit, for example, the AdvanceBio AAA kit (Agilent) or the AccQ-Tag AA kit (Waters).

The methods described herein may further comprise determining the concentration of each component amino acid of the polypeptide, for example, through comparison of with a standard. The comparison may be done using a chromatographic method, such as an HPLC, GCMS, or LCMS, or other detection method, such as NMR.

Once the concentration of each component amino acid is known, the concentration of the intact conjugated polypeptide may be extrapolated. In an embodiment, the methods described herein comprise comparing the concentration of each component amino acid in a sample with the known sequence of the polypeptide. For example, the methods may be applied to a polypeptide with a known amino acid sequence of known length n, e.g., X₁, X₂, X₃, X₄, X₅, X₆, Xₙ, wherein X can be any amino acid, and X at each numbered position may be different or the same than any other numbered position(s), and R_{D} is the number of residues of each distinct amino acid in the polypeptide. In an embodiment, the total molar concentration of the polypeptide in a hydrolyzed sample may be calculated by the following approach: (i) the total molar concentration TC_{D} is determined for each distinct amino acid in the polypeptide; (ii) each TC_{D} is divided by the R_{D} for that amino acid to obtain the average molar concentration AC_{D} for that amino acid; and (iii) the sum of all of the AC_{D} is divided by n to obtain the concentration of total polypeptide in sample (μmol/mL).

The methods detailed herein may also be used to evaluate the impurity profile of a polymer composition. In an embodiment, the method further comprises acquiring the concentration of unconjugated (e.g., free) polypeptide in the polymer composition.

In another aspect, the present disclosure features methods for evaluating a modified polymer comprising a polypeptide within a polymer composition that do not entail subjecting the polymer composition to degradative conditions, e.g., hydrolytic conditions. For example, evaluating may comprise performing a non-degradative analysis method such as refractometry or spectroscopy on a sample of the composition. In an embodiment, non-degradative methods of evaluating may include determining the overall concentration of a modified polymer in the sample, determining the concentration of a polypeptide bound to a modified polymer in the sample, identifying the sequence of a polypeptide bound to a modified polymer in the sample, or querying the polymer composition for certain impurities. In an embodiment, evaluating the polymer composition (e.g., in a non-degradative manner) comprises acquiring a value of the refractive index of the polymer composition, e.g., in a solid or liquid sample.

In order to use a refractive index to evaluate a polymer composition, e.g., described herein, the polymer composition may be dissolved or suspended in an aqueous medium, e.g., saline or other buffer (e.g., HEPES, sodium phosphate, Tris, or PBS). The refractive index of the polymer composition may be compared to a control sample, for example, comprising only the aqueous medium and not the polymer composition. In an embodiment, all samples are analyzed at the same wavelength of light and temperature.

In an embodiment, a specific refractive index increment value for each sample is determined (i.e., a “dn/dc value”). The specific refractive index increment is related to the amount by which the refractive index of a polymer composition changes with respect to another parameter, e.g., the amount of a polypeptide in the composition (e.g., conjugated polypeptide or unconjugated polypeptide). For example, the specific refractive index increment may be dependent on the concentration of the polymer (e.g., modified polymer) in the sample. In an embodiment, specific refractive index increment is provided as any unit, e.g., g/mol or (1/% w/w). For example, for a polymer composition comprising a GRGDSP-modified alginate, a specific refractive index increment may be determined for each of the GRGDSP moiety and a representative component of the alginate, such as a guluronic acid moiety. Exemplary methods are provided herein in Example 5.

The refractive index (nD) of a polymer composition may be between 1.3300 and 1.3400, e.g., at a specific temperature or temperature range (e.g., between about 2-8° C., or about 22-28° C.). For example, the refractive index of a polymer composition described herein may be between 1.3310 and 1.3400, e.g., 1.3320 and 1.3400, 1.3330 and 1.3400, 1.3340 and 1.3400, 1.3360 and 1.3400, 1.3370 and 1.3400, and 1.3380 and 1.3400. In an embodiment, the refractive index of a polymer composition is between 1.3350 and 1.3400.

### Enumerated Embodiments

1. A method of evaluating a polymer composition comprising a polymer modified with a polypeptide, the method comprising:

(a) subjecting the polymer composition to reaction conditions that allow for:
(i) release of the polypeptide from the modified polymer; and/or
(ii) hydrolysis of the polypeptide into component amino acids;

thereby evaluating the polymer composition.

2. The method of embodiment 1, wherein the method comprises (i).
3. The method of any one of embodiments 1 or 2, wherein the method comprises (ii).
4. The method of any one of embodiments 1-3, wherein the method further comprises:

(b) acquiring a value for the concentration of each component amino acid of the polypeptide.

5. The method of any one of embodiments 1-4, wherein the method further comprises:

(c) acquiring a value for the concentration of the polypeptide bound to the modified polymer.

6. The method of embodiment 5, wherein the acquiring comprises using the value obtained in step (b).
7. The method of any one of embodiments 1-6, wherein the polymer in the modified polymer is a polysaccharide.
8. The method of embodiment 7, wherein the polymer in the modified polymer is an alginate.
9. The method of embodiment 8, wherein the alginate has an average molecular weight of 75 kD to 150 kD.
10. The method of any one of embodiments 8-9, wherein the alginate has a guluronate to mannuronate (G:M) ratio of greater than or equal to 1.5.
11. The method of any one of embodiments 1-10, wherein the polypeptide is covalently bound to the polymer (e.g., alginate).
12. The method of any one of embodiments 1-10, wherein the polypeptide is non-covalently bound to the polymer (e.g., alginate).
13. The method of any one of embodiments 1-10, wherein the polypeptide is covalently bound to the polymer (e.g., alginate) through a linker (e.g., an amino acid linker).
14. The method of embodiment 13, wherein the linker comprises at least one glycine residue (e.g., at least 2, 3, or 4 glycine residues).
15. The method of any one of embodiments 1-14, wherein the polymer composition or comprises a single type of modified polymer (e.g., modified alginate).
16. The method of any one of embodiments 1-14, wherein the polymer composition comprises a plurality of modified polymers (e.g., at least two modified polymers, at least three modified polymers).
17. The method of any one of embodiments 1-16, wherein the polypeptide comprises a cell-binding polypeptide (CBP).
18. The method of embodiment 17, wherein the CBP comprises a sequence selected from RGD, RGDSP, DGEA, FYFDLR, PHSRN, YIGSR, and a sequence listed in Table 1.
19. The method of any one of embodiments 17 or 18, wherein the CBP comprises a plurality of sequences selected from RGD, RGDSP, DGEA, FYFDLR, PHSRN, YIGSR, and a sequence listed in Table 1.
20. The method of embodiment 11, wherein the polypeptide covalently bound to the modified polymer (e.g., modified alginate) is a linker-CBP.
21. The method of embodiment 20, wherein the linker-CBP comprises GRGD or GRGDSP.
22. The method of any one of embodiments 1-21, wherein the modified polymer comprises alginate-GRGD or alginate-GRGDSP.
23. The method of any one of embodiments 1-22, wherein the modified polymer comprises alginate-GRGD.
24. The method of any one of embodiments 1-22, wherein the modified polymer comprises alginate-GRGDSP.
25. The method of any one of embodiments 1-24, wherein the reaction conditions in step (a) comprise contacting the polymer composition with an acidic solution (e.g., an HCl solution, e.g., a 1N-8N HCl solution).
26. The method of any one of embodiments 1-24, wherein the reaction conditions in step (a) comprise heating the polymer composition (e.g., at a temperature greater than about 25° C., 40° C., 60° C., 80° C., 100° C., 120° C., or higher).
27. The method of any one of embodiments 1-26, wherein the reaction conditions in step (a) comprise exposing the polymer composition to microwave irradiation.
28. The method of any one of embodiments 1-27, wherein step (b) comprises a separation step.
29. The method of embodiment 28, wherein the separation step comprises chromatography (e.g., size-exclusion chromatography, ion-exchange chromatography, gel filtration chromatography, reversed-phase chromatography, or hydrophobic interaction chromatography).
30. The method of any one of embodiments 1-29, wherein acquiring a value for the concentration in step (b) comprises determining the area of a chromatogram peak for each component amino acid of the polypeptide.
31. The method of embodiment 30, wherein acquiring a value for the concentration in step (b) further comprises comparing the area of a chromatogram peak for each component amino acid of the polypeptide with a standard, e.g., to determine the concentration of each component amino acid of the polypeptide.
32. The method of embodiment 31, wherein the standard comprises a mixture of amino acids.
33. The method of any one of embodiments 1-32, further comprising modifying the individual amino acids prior step (b), e.g., prior to chromatographic separation.
34. The method of embodiments 33, wherein modifying the individual amino acids comprises derivatization with a derivatizing agent.
35. The method of embodiment 34, wherein the derivatizing agent comprises ortho-phthaladehyde (OPA) or 9-fluoroenylmethyl chloroformate (Fmoc).
36. The method of any one of embodiment 1-35, further comprising acquiring a value for the concentration of free polypeptide (i.e., unconjugated polypeptide) in the polymer composition.
37. The method of embodiment 36, wherein acquiring a value for the concentration of free polypeptide (i.e., unconjugated polypeptide) in the polymer composition comprises:

(a′) separating the polymer composition into a polymer bound fraction and a non-polymer bound fraction.

38. The method of any one of embodiments 36-37, further comprising

(b′) retaining the non-polymer bound fraction.

39. The method of any one of embodiments 36-38, further comprising:

(c′) acquiring a value for the concentration of the polypeptide in the non-polymer bound fraction.

40. The method of any one of embodiments 37-39, wherein the separating of step (a′) comprises filtration, e.g., with a molecular weight cutoff filter.
41. The method of any one of embodiments 39-40, wherein step (c′) comprises a separation step.
42. The method of embodiment 41, wherein the separation step comprises chromatography (e.g., size-exclusion chromatography, ion-exchange chromatography, gel filtration chromatography, reversed-phase chromatography, or hydrophobic interaction chromatography).
43. The method of any one of embodiments 39-42, wherein acquiring a value for the concentration of step (c′) further comprises determining the area of a chromatogram peak for polypeptide of the non-polymer bound fraction.
44. The method of embodiment 43, wherein acquiring the concentration of step (c′) further comprises comparing the area of a chromatogram peak for polypeptide of the non-polymer bound fraction with a standard, e.g., to determine the concentration of polypeptide of the non-polymer bound fraction.
45. The method of embodiment 44, wherein the standard comprises a single peptide or single polypeptide.
46. The method of any one of embodiments 1-45, wherein evaluating a polymer composition comprises determining the concentration of polypeptide conjugated (e.g., covalently bound) to a modified polymer in the polymer composition.
47. The method of any one of embodiments 1-46, wherein each polymer in the polymer composition does not comprise an amide bond (e.g., each polymer is not a peptide, polypeptide, or protein).
48. The method of any one of embodiments 1-47, wherein determining the concentration of polypeptide conjugated (e.g., covalently bound) to a modified polymer in the polymer composition comprises:

(a″) acquiring a value for the total concentration of polypeptide in the polymer composition or semi-permeable device.

49. The method of embodiment 48, further comprising:

(b″) acquiring a value for the concentration of free polypeptide (i.e., unconjugated polypeptide) in the polymer composition.

50. The method of any one of embodiments 48-49, further comprising:

(c″) subtracting the value of the concentration of free polypeptide (e.g., as determined in step (b″)) from the value of the total concentration of polypeptide in the polymer composition.

51. A method of evaluating a polymer composition comprising an alginate modified with a cell-binding polypeptide (CBP), the method comprising:

(a) subjecting the polymer composition to reaction conditions that allow for:
(i) release of the CBP from the modified alginate; and/or
(ii) hydrolysis of the CBP into component amino acids;

thereby evaluating the polymer composition.

52. The method of embodiment 51, wherein the method comprises (i).
53. The method of any one of embodiments 51-52, wherein the method comprises (ii).
54. The method of any one of embodiments 51-53, wherein the method further comprises:

(b) acquiring a value for the concentration of each component amino acid of the CBP.

55. The method of any one of embodiments 51-54, wherein the method further comprises:

(c) acquiring a value for the concentration of the CBP bound to the modified alginate.

56. The method of embodiment 55, wherein the acquiring comprises using the value obtained in step (b).
57. The method of any one of embodiments 51-56, wherein the alginate has an average molecular weight of 75 kD to 150 kD.
58. The method of any one of embodiments 51-57, wherein the modified alginate has a guluronate to mannuronate (G:M) ratio of greater than or equal to 1.5.
59. The method of any one of embodiments 517-58, wherein the CBP is covalently bound to the modified alginate, e.g., through a linker (e.g., an amino acid linker).
60. The method of embodiment 59, wherein the linker comprises at least one glycine residue (e.g., at least 2, 3, or 4 glycine residues).
61. The method of any one of embodiments 51-60, wherein the CBP comprises a sequence selected from RGD, RGDSP, DGEA, FYFDLR, PHSRN, YIGSR, and a sequence listed in Table 1.
62. The method of any one of embodiments 51-60, wherein the CBP comprises a plurality of sequences selected from RGD, RGDSP, DGEA, FYFDLR, PHSRN, YIGSR, and a sequence listed in Table 1.
63. The method of any one of embodiments 51-62, wherein the CBP comprises GRGD or GRGDSP.
64. The method of any one of embodiments 51-63, wherein the CBP comprises GRGD.
65. The method of any one of embodiments 51-63, wherein the CBP comprises GRGDSP.
66. A method of determining the concentration of a cell-binding polypeptide (CBP) bound to a polymer, the method comprising:

(a) acquiring a value for the concentration of the total CBP in the polymer composition.

67. The method of embodiment 66, wherein the method further comprises:

(b) acquiring a value for the concentration of the free (i.e., unconjugated) CBP in the polymer composition.

68. The method of any one of embodiments 66-67, wherein the method further comprises:

(c) subtracting the value of the concentration of free CBP (e.g., as determined in step (b)) from the value of the total CBP concentration in the polymer composition.

69. The method of any one of embodiments 67-68, wherein step (a) is performed prior to step (b).
70. The method of any one of embodiments 67-68, wherein step (b) is performed prior to step (a).
71. The method of any one of embodiments 66-70, wherein step (a) comprises:

(i) subjecting the polymer composition to reaction conditions that allow for:
(I) release of the CBP from the polymer; and/or
(II) hydrolysis of the CBP into component amino acids.
   72. The method of embodiment 71, further comprising:

(ii) acquiring a value for the concentration of each component amino acid of the CBP.

73. The method of any one of embodiments 71-72, further comprising:

(iii) using the value obtained in step (ii), acquiring a value for the concentration of the CBP bound to the modified polymer.

74. A method of evaluating a polymer composition comprising a polymer modified with a polypeptide, the method comprising:

(a) acquiring a value for the refractive index of the polymer composition.

75. The method of embodiment 74, further comprising:

(b) acquiring a value of the total polypeptide conjugated (e.g., covalently bound) to the modified polymer in the polymer composition.

76. The method of any one of embodiments 74-75, further comprising:

(c) acquiring a value for the concentration of the polymer modified with the polypeptide.

77. The method of embodiment 76, wherein acquiring comprises using the values obtained in each of steps (a) and (b).
78. The method of any one of embodiments 74-77, wherein the polymer in the modified polymer is a polysaccharide.
79. The method of embodiment 78, wherein the polymer in the modified polymer is an alginate.
80. The method of embodiment 79, wherein the modified alginate has an average molecular weight of 75 kD to 150 kD.
81. The method of any one of embodiments 79-80, wherein the alginate has a guluronate to mannuronate (G:M) ratio of greater than or equal to 1.5.
82. The method of any one of embodiments 74-81, wherein the polypeptide is covalently bound to the polymer (e.g., alginate).
83. The method of any one of embodiments 74-81, wherein the polypeptide is non-covalently bound to the polymer (e.g., alginate).
84. The method of any one of embodiments 74-81, wherein the polypeptide is covalently bound to the polymer (e.g., alginate) through a linker (e.g., an amino acid linker).
85. The method of embodiment 84, wherein the linker comprises at least one glycine residue (e.g., at least 2, 3, or 4 glycine residues).
86. The method of any one of embodiments 74-85, wherein the polypeptide comprises a cell-binding polypeptide (CBP).
87. The method of embodiment 86, wherein the CBP comprises a sequence selected from RGD, RGDSP, DGEA, FYFDLR, PHSRN, YIGSR, and a sequence listed in Table 1.
88. The method of embodiment 86, wherein the CBP comprises a plurality of sequences selected from RGD, RGDSP, DGEA, FYFDLR, PHSRN, YIGSR, and a sequence listed in Table 1.
89. The method of embodiment 86, wherein the polypeptide covalently bound to the modified polymer (e.g., modified alginate) is a linker-CBP.
90. The method of embodiment 89, wherein the linker-CBP comprises GRGD or GRGDSP.
91. The method of any one of embodiments 89-90, wherein the linker-CBP comprises alginate-GRGD.
92. The method of any one of embodiments 89-90, wherein the linker-CBP comprises alginate-GRGDSP.
93. The method of any one of claims 74-92, wherein acquiring the value of the refractive index of step (a) comprises acquiring a refractometer reading (nD) at a specific wavelength and/or specific temperature.
94. The method of any one of embodiments 74-93, wherein acquiring a value of the total polypeptide conjugated (e.g., covalently bound) to a modified polymer in the polymer composition of step (b) comprises:

(b′) subjecting the polymer composition to reaction conditions that allow for:
(i) release of the polypeptide from the modified polymer; and
(ii) hydrolysis of the polypeptide into component amino acids

(b″) acquiring a value for the concentration of each component amino acid of the polypeptide; and

(b′″) using the value obtained in step (b″), acquiring a value for the concentration of the polypeptide bound to the modified polymer.

95. The method of embodiment 94, further comprising acquiring a dn/dc value for a component of the polymer composition (e.g., a component of the modified polymer).
96. The method of embodiment 95, further comprising using the dn/dc value in step (c) of the method, e.g., to acquire a value for the concentration of the polymer modified with the polypeptide
97. A method of determining the concentration of a cell-binding polypeptide (CBP) bound to a polymer (i.e., a CBP-modified polymer) in a polymer composition, the method comprising:

(a) acquiring a value for the refractive index of the polymer composition;

(b) acquiring a value of the total CBP conjugated (e.g., covalently bound) to the polymer in the polymer composition;

(c) using the values obtained in each of steps (a) and (b), acquiring a value for the concentration of the CBP-modified polymer;

thereby determining the concentration of a cell-binding polypeptide (CBP) bound to the polymer in a polymer composition.

98. The method of embodiment 97, wherein the polymer is an alginate.
99. The method of embodiment 98, wherein the alginate has an average molecular weight of 75 kD to 150 kD.
100. The method of any one of embodiments 97-99, the CBP is covalently bound to the modified alginate, e.g., through a linker (e.g., an amino acid linker).
101. The method of embodiment 100, wherein the linker comprises at least one glycine residue (e.g., at least 2, 3, or 4 glycine residues).
102. The method of any one of embodiments 97-101, wherein the CBP comprises a sequence selected from RGD, RGDSP, DGEA, FYFDLR, PHSRN, YIGSR, and a sequence listed in Table 1.
103. The method of any one of embodiments 97-101, wherein the CBP comprises a plurality of sequences selected from RGD, RGDSP, DGEA, FYFDLR, PHSRN, YIGSR, and a sequence listed in Table 1.
104. The method of any one of embodiments 97-103, wherein the CBP comprises GRGD or GRGDSP.
105. The method of any one of embodiments 97-104, wherein the CBP comprises GRGD.
106. The method of any one of embodiments 97-104, wherein the CBP comprises GRGDSP.
107. The method of any one of embodiments 1-106, wherein the level of free polypeptide is less than 0.01 umol/g, e.g., as determined by % weight of polymer in the composition.
108. A polymer composition comprising a polymer modified with a polypeptide, wherein the concentration of the polypeptide is between 0.1 umol/g and 1.0 umol/g, e.g., as determined by % weight of polymer in the composition, e.g., based on a method of any one of embodiments 1-107.
109. A polymer composition comprising a polymer modified with a CBP, wherein the concentration of the CBP is between 0.1 umol/g and 1.0 umol/g, e.g., as determined by % weight of polymer in the composition, e.g., based on a method of any one of embodiments 1-107.
110. A polymer composition comprising an alginate polymer modified with a CBP (e.g., RGD, RGDSP, DGEA, FYFDLR, PHSRN, YIGSR, GRGDSP, and a sequence listed in Table 1), wherein the concentration of the CBP is between 0.1 umol/g and 1.0 umol/g, e.g, as determined by % weight of alginate polymer in the composition, e.g., based on a method of any one of embodiments 1-107.
111. An alginate polymer modified with a CBP, wherein the concentration of the CBP is between 0.1 umol/g and 1.0 umol/g, e.g., as determined by % weight of alginate polymer in the composition, e.g., based on a method of any one of embodiments 1-107.
112. An alginate polymer modified with a CBP selected from RGD, RGDSP, DGEA, FYFDLR, PHSRN, YIGSR, GRGDSP, and a sequence listed in Table 1, wherein the concentration of the CBP is between 0.1 umol/g and 1.0 umol/g, e.g., as determined by % weight of alginate polymer in the composition, e.g., based on a method of any one of embodiments 1-107.

### EXAMPLES

In order that the disclosure described herein may be more fully understood, the following examples are set forth. The examples described in this application are offered to illustrate the devices (e.g., capsules, particles, chemical modifications, compositions and methods provided herein and are not to be construed in any way as limiting their scope.

### Example 1. Preparation of Exemplary Modified Polymers

A polymeric material may be covalently modified with a polypeptide prior to formation of a semi-permeable device (e.g., a hydrogel capsule) using methods known in the art, see, e.g., Jeon O, et al., Tissue Eng Part A. 16:2915-2925 (2010) and Rowley, J. A. et al., Biomaterials 20:45-53 (1999).

For example, in the case of alginate, an alginate solution (1%, w/v) was prepared with 50 mM of 2-(N-morpholino)-ethanesulfonic acid hydrate buffer solution containing 0.5 M NaCl at pH 6.5, and sequentially mixed with N-hydroxysuccinimide and 1-ethyl-3-[3-(dimethylamino)-propyl] carbodiimide (EDC). The molar ratio of N-hydroxysuccinimide to EDC was 0.5:1.0. The polypeptide of interest was added to the alginate solution. The amounts of polypeptide and coupling reagent added depends on the desired concentration of the polypeptide bound to the alginate. By increasing the amount of peptide and coupling reagent, a higher polypeptide concentration can be obtained. After reacting for 24 h, the reaction was purified by dialysis against ultrapure deionized water (diH2O) (MWCO 3500) for 3 days, treated with activated charcoal for 30 min, filtered (0.22 mm filter), and concentrated to the desired viscosity.

Various alginate solutions containing a CBP-alginate or a mixture of a cell-binding protein and an unmodified alginate were prepared as summarized in Table 3 below.

TABLE 3
Experimental Alginate Solutions Containing Cell-Binding
Sites Used To Form Cell-Containing Compartments

| | Cell Binding | | |
|---|---|---|---|
| Solution Name* | Peptide (CBP) | Linker | Solution Composition**, *** |
| VLVG-4GRGDSP | RGDSP | GGGG | 5 w/v % of CBP-alginate blended at |
| | | | 70:30 volume ratio with 3% afibrotic |
| | | | compound in saline |
| MVG-GRGDSP | RGDSP | G | 1.4 w/v % of CBP-alginate in saline |
| 1G-MVG: 0.71, 143 cP | RGDSP | G | Viscosity = 143 cP |
| 1G-MVG: 0.71, 79 cP | | | Viscosity = 79 cP |
| 1G-MVG: 0.71, 97 cP | | | Viscosity = 97 cP |
| 1G-MVG: 0.71, 202 cP | | | Viscosity = 202 cP |
| 1G-SLG100: 0.65 | RGDSP | G | Viscosity = 98 cP |
| 1G-RGD: 0.22 | RGDSP | G or | Viscosity = 118 cP |
| 1G-RGD: 0.41 | | | Viscosity = 57 cP |
| 1G-RGD: 0.33 | | | Viscosity = 113 cP |
| 1G-RGD: 0.72 | | | Viscosity = 133 cP |
| 1G-RGD: 1.47 | | | Viscosity = 141 cP |
| 4G-RGD: 0.53 | | | Viscosity = 70 cP |
| 4G-RGD: 0.96 | | | Viscosity = 58 cP |
| 4G-RGD: 1.77 | | | Viscosity = 102 cP |
| 4G-RGD: 2.95 | | | Viscosity = 123 cP |
| 1G-RGD: 6.04 | RGDSP | G | Viscosity = 102 cP |
| 1G-RGD: 6.04, 1:55 | | | Blended 1:55 with 1.4 w/v % SLG20 |
| 1G-RGD: 6.04, 1:30 | | | Blended 1:30 with 1.4 w/v % SLG20 |
| 1G-RGD: 6.04, 1:15 | | | Blended 1:15 with 1.4 w/v % SLG20 |
| 4G-DGEA: 0.15 | DGEA | GGGG | Viscosity = 133 cP |
| 4G-PHSRN: 0.68 | PHSRN | GGGG | Viscosity = 133 cP |
| G-PHSRN: 0.83 | PHSRN | G | Viscosity = 108 cP |
| PHSRN/RGD, same | RGDSP and | G | Viscosity = 98 cP |
| polymer | PHSRN | | |
| G-HAVDI: 0.38 | HAVDI | G | Viscosity = 132 cP |
| Collagen I | Full-length protein | None | 0.36 mg/ml Collagen I in 1.4 w/v % |
| | | | SLG20 in saline |
| Hyaluronic Acid (HA) | Full-length | None | 0.15 w/v % HA in 1.4 w/v % SEG20 in |
| | glycosaminoglycan | | saline |

*Where known, the solution names incorporate the polypeptide concentration level as percent nitrogen determined by combustion analysis.
**When present, the type and concentration of cells are noted in the Examples below.
***SLG20 refers to the type of alginate used in these studies

### Example 2. Determining Total CBP in a CBP-Modified Alginate Sample

To determine the total amount of CBP (including both free CBP and conjugated CBP) in a sample of CBP-modified polymer, the CBP-modified polymer may be subjected to acid hydrolysis to cleave the CBP into individual amino acids. The individual amino acids may subsequently be derivatized (e.g., by automated pre-column on-line derivatization), using derivatizing agents (e.g., ortho-phthalaldehyde (OPA) in the case of primary amino acids, or 9-fluoroenylmethyl chloroformate (Fmoc) in the case of secondary amino acids). The derivatized amino acids may then be immediately separated and analyzed by liquid chromatography. Here, the total CBP was quantified in a sample of CBP-modified alginate as described below.

Acid hydrolysis of peptide-modified alginate sample. Approximately 12-16 mg of solid lyophilized CBP-modified alginate (GRGDSP-conjugated alginate), or 1000±50 mg of GRGDSP-conjugated alginate in saline solution, was weighed into a reaction vial, ensuring the solid remained at the bottom of the vial. 6N HCl (10 mL) solution was then added to the vial with a stir bar. The sealed mixture was heated to 120° C. and stirred at 400 rpm for 6 hours, then cooled to ambient temperature. The entire solution was then transferred to a 20 mL volumetric flask, and the empty reaction vial was rinsed thoroughly three times with 2 mL of LCMS grade water and transferred to the volumetric flask using the same pipet to ensure complete transfer of the sample. The volumetric flask was then brought to volume with LCMS grade water and inverted to mix the solution well. The mixture was transferred to a 50 mL centrifuge tube and centrifuged at 5000 rpm for 10 min. 1 mL of the supernatant was then transferred to an LC vial and stored at 2-8° C. overnight. The next day, the sample was heated to 60° C. and completely dried by placing a needle delivering a nitrogen stream directly over the sample (ensuring the needle did not touch the sample). Once dried, 0.25 mL of the 0.1 μmol/mL internal standard mixture (see preparation below) was added to the LC vial, the solution was vortexed thoroughly, then transferred to a second LC vial with a 0.25 mL insert. The sample was stored at 2-8° C. until use.

Alternatively, hydrolysis of the CBP-modified alginate (GRGDSP-conjugated alginate) was carried out by microwave irradiation. Briefly, 1000±50 mg of GRGDSP-conjugated alginate in saline solution was weighed into a microwave vial. 6N HCl (10 mL) solution was then added to the vial with a stir bar, and the vial was capped and placed into a microwave autosampler. Each sample was processed according to the following conditions:

| | | |
|---|---|---|
| Parameter | Setting | Stir RPM |

| | | |
|---|---|---|
| Ramp Time | AFAP | 400 |

| | | | |
|---|---|---|---|
| Hold Temp | 152° | C. | 400 |
| Hold Time | 20 | min | 400 |
| Cooling Temp | 55° | C. | 400 |

Each vial was removed and the contents were transferred into a volumetric flask, to which 2 mL of LCMS grade water was added (2×). The flask was capped and inverted to mix, then the contents were transferred to a 50 mL centrifuge tube. The sample was centrifuged at 5000 rpm for approximately 10 minutes. 1 mL of the supernatant was then transferred to an LC vial and stored at 2-8° C. overnight. The next day, the sample was heated to 60° C. and completely dried by placing a needle delivering a nitrogen stream directly over the sample (ensuring the needle did not touch the sample). Once dried, 0.25 mL of the 0.1 μmol/mL internal standard mixture (see preparation below) was added to the LC vial, the solution was vortexed thoroughly, then transferred to a second LC vial with a 0.25 mL insert. The sample was stored at 2-8° C. until use.

Amino acid standard stock solutions. A standard solution was prepared for each amino acid using the following procedure. Each amino acid (ca. 0.5 mmol) was weighed into a 50 mL volumetric flask. The volumetric flask was then brought to volume with 0.1N HCl, and the solution was mixed well by inverting or vortexing. Samples were stored at 2-8° C. The exact amounts of amino acids and the final concentrations obtained are provided in Table 4 below.

TABLE 4
Amino acid stock solutions.

| | | | Actual | | | Actual |
|---|---|---|---|---|---|---|
| Letter | Full | MW | weight | Volume | Purity | Concentration |
| name | name | (g/mol) | (mg) | (mL) | (%) | (μmol/mL) |
| D | Aspartic acid | 133.10 | 66.38 | 50 | 100 | 9.97 |
| S | Serine | 105.09 | 52.55 | 50 | 100 | 10.00 |
| G | Glycine | 75.07 | 38.56 | 50 | 100 | 10.27 |
| R | Arginine HCl | 210.66 | 102.67 | 50 | 100 | 9.75 |
| N-iSTD | Norvaline | 117.15 | 58.76 | 50 | 100 | 10.03 |
| S-iSTD | Sarcosine | 89.09 | 44.00 | 50 | 98.4 | 9.72 |
| P | Proline | 115.13 | 57.74 | 50 | 100 | 10.03 |

Preparation of internal standards. A 1 μmol/mL internal standard solution was prepared by adding 1.0 mL of the norvaline stock solution (NiSTD) and 1.0 mL of the sarcosine stock solution (SiSTD) to the same 10 mL volumetric flask to prepare a norvaline/sarcosine stock solution (NiSTD-SiSTD). The volumetric flask was then brought to volume using 0.1N HCl and mixed well by inverting and vortexing. A 0.1 μmol/mL internal sample solution was then prepared by adding 1.0 mL of the 1 μmol/mL NiSTD-SiSTD solution to a 10 mL volumetric flask, and bringing the volumetric flask to volume with 0.1N HCl, followed by mixing by inverting or vortexing. Both NiSTD-SiSTD internal standard solutions were stored at 2-8° C. until use.

Preparation of 5× amino acids/internal standard mixtures (5AA+iSTD solution). Three separate standard solutions containing an internal standard and a mixture of 5 amino acids at varying concentrations (0.025 μmol/mL, 0.1 μmol/mL, and 0.25 μmol/mL) were prepared using the following procedure. Using the amounts provided in Table 5 below, the stock solutions of aspartic acid (D), serine (S), glycine (G), arginine (R), and proline (P) were each added to 3 10 mL volumetric flasks, along with either NiSTD or sarcosine SiSTD stock solutions, using the volumes indicated in Table 5. The volumetric flasks were then brought to volume with 0.1N HCl and mixed well by inverting or vortexing, to provide three standard solutions with the final concentrations indicated in Table 5. Each sample was stored at 2-8° C.

TABLE 5
Mixed amino acid/internal standard solutions prepared.

| D/S/G/R/P | D/S/G/R/P | N-iSTD or | N-iSTD or | | D/S/G/R/P | iSTD |
|---|---|---|---|---|---|---|
| STD | STD | S-iSTD | S-iSTD | Total | final | final |
| conc. | volume | conc. | volume | volume | conc. | conc |
| (μmol/mL) | (μL) | (μmol/mL) | (μL) | (mL) | (μmol/mL) | (μmol/mL) |
| 10 | 25 | 10 | 100 | 10 | 0.025 | 0.1 |
| 10 | 100 | 10 | 100 | 10 | 0.1 | 0.1 |
| 10 | 250 | 10 | 100 | 10 | 0.25 | 0.1 |

Preparation of 17× amino acids/internal standard mixture (17AA+iSTD solution). Exactly 0.9 mL of a commercially available 17 amino acid standard solution (0.1 μmol/mL; Agilent 5061-3332) was transferred to an LC vial. Into the same vial was then added 100 μL of the 1 μmol/mL NiSTD-SiSTD solution (see above). The resulting solution was then mixed by vortexing, and an aliquot of 100 μL was subsequently transferred to an LC vial with an 0.25 mL insert. The sample was stored at 2-8° C.

Derivatization of Amino Acids. Automated pre-column on-line derivatization was used to derivatize amino acids immediately prior to injection on the LC, which was set to the following sequence. First, borate buffer (2.5 μL; Agilent 5061-3339) was drawn from “location 1,” then 1 μL of sample was drawn, and the resulting 3.5 μL aliquot was mixed from the seat with the default speed 5 times, and paused for 0.2 min. Then 0.5 μL of OPA reagent (Agilent 5061-3335) was drawn from “location 2,” and the resulting 4 μL aliquot was mixed from the seat with default speed 10 times. Next, 0.4 μL of Fmoc reagent (Agilent 5061-3337) was drawn from “location 3,” and the resulting 4.4 μL aliquot was mixed from seat with default speed 10 times. Then injection diluent (32 μL) was drawn from “location 4,” and the final aliquot was mixed with default speed for 8 times, and injected into the LC.

HPLC conditions. HPLC was performed with an Agilent 1260 LC equipped with both a UV and fluorescence detector, using an AdvanceBio AAA LC, 2.7 μm, 4.6×100 mm (Agilent 655950-802) column. The mobile aqueous phase used was a solution of 10 mM Na₂HPO₄ and 10 mM Na₂B₄O₇ (pH 8.2), and the mobile organic phase was a 45/45/10 mixture of acetonitrile/methanol/water, at a flow rate of 1.5 mL/min. The column temperature was at 40° C.

The UV detector was set to a wavelength of 338 nm. At approximately 11 minutes, following the last eluting OPA-derivatized amino acid (lysine) and before the first eluting Fmoc-derivatized amino acid (hydroxyproline), the UV detector wavelength was switched to 262 nm. The fluorescence detector was set to an excitation wavelength of 340 nm and an emission wavelength of 450 nm. After the last eluting OPA-derivatized amino acid (lysine) and before the first eluting Fmoc-derivatized amino acid (hydroxyproline), at approximately 11 minutes, the fluorescence detector was switched to an excitation wavelength of 260 nm and an emission wavelength of 325 nm.

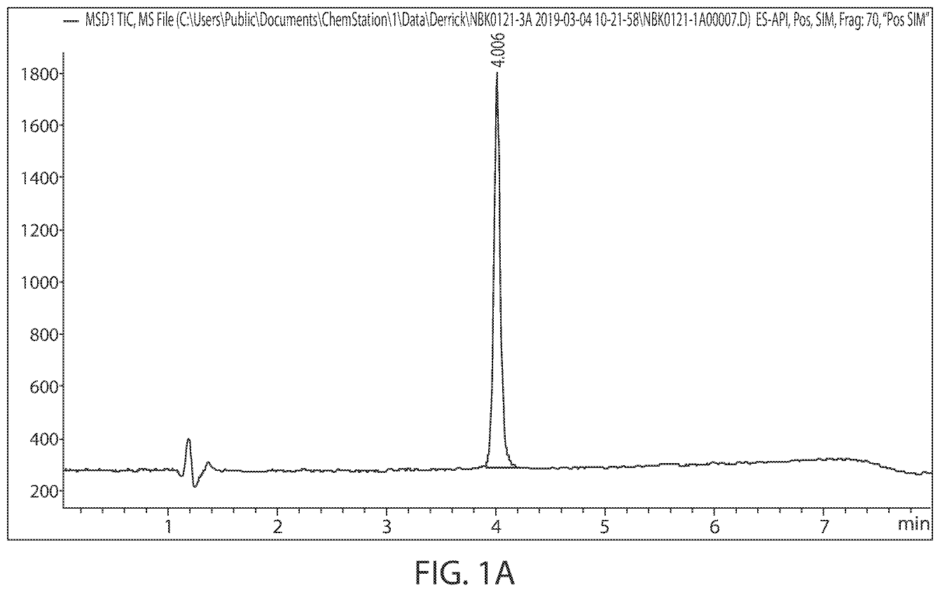

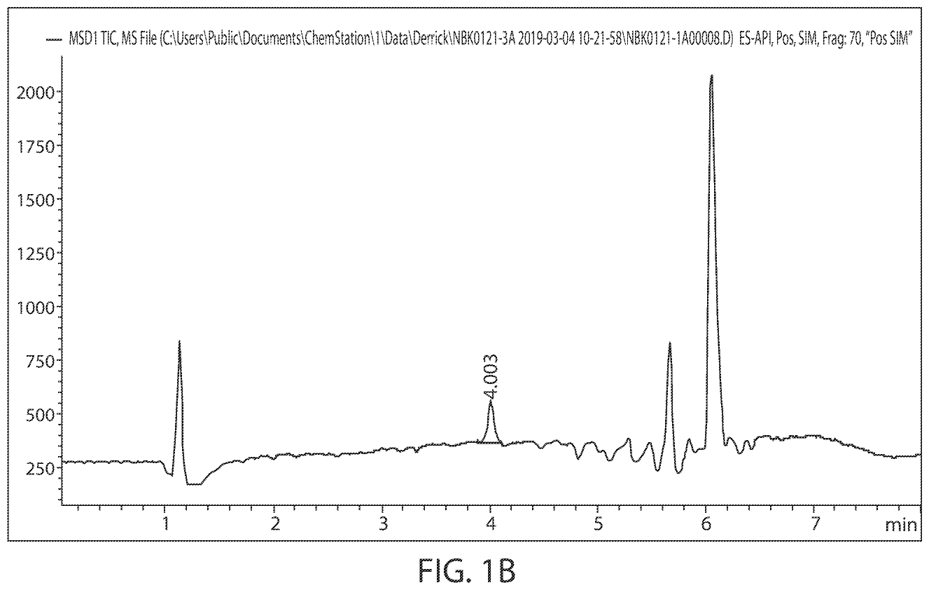

Sample runs were carried out with the set sequence as follows. A blank of 0.1N HCl was run 5 times, followed by 3 runs each of 0.025 μmol/mL, 0.1 μmol/mL, and 0.25 μmol/mL 5AA+iSTD solutions. The blank (0.1N HCl) was then run twice more, followed by the 0.1 μmol/mL 17AA (+iSTD) standard, then followed by a further run of the blank (0.1N HCl). Then, acid-hydrolyzed samples were run in sequence, and every 10 samples was bracketed by injection with the 0.1 μmol/mL 5AA+iSTD solution. Finally, each of the 0.025 μmol/mL, 0.1 μmol/mL, and 0.25 μmol/mL 5AA+iSTD solutions were injected in that order, followed by flushing the column. Exemplary HPLC chromatograms can be found in FIGS. 1A-B, showing an overlay of the 3 UV chromatograms of the 17AA standard mixtures at each concentration (FIG. 1A), and an overlay of the fluorescence chromatograms of the 17AA standard mixtures at each concentration (FIG. 1B).

System suitability criteria. Data was confirmed against the suitability criteria as follows: no significant interferences in the UV or fluorescence chromatograms of blanks; no more than 5% RSD of retention time in the initial three injections; no more than 20% RSD of area in the initial three injections; no more than 20% RSD of relative area in the initial three injections; no more than 5% RSD of retention time in all injections; no more than 20% RSD of area in all injections; no more than 20% RSD of relative area in all injections; a resolution showing baseline separation of glycine from other amino acids, norvaline (NiSTD) from other amino acids, and sarcosine (SiSTD) from proline; a linearity with a coefficient of determination no less than 0.99; and a quantitated result within 80 to 120% of theoretical.

HPLC data analysis and calculations. The amino acid peaks were identified by matching the retention time of the amino acid peaks in the sample with the peaks in the 17AA+iSTD solution and the 5AA+iSTD solution, in both UV and fluorescence chromatograms.

The relative area of aspartic acid, serine, glycine, or arginine can be calculated with the following formula: relative area (D, S, G, or R)=area (D, S, G, or R)/area (norvaline).

The relative area of proline can be calculated with the following formula: relative area (P)=area (P)/area (sarcosine).

Preparation of standard calibration curve. Concentrations of standard solutions were calculated based on reported value from certificate of analysis or based on actual weights adjusted by dilution factor during the standard preparation. The average area or average relative area vs. concentration for each amino acid of interest was then plotted from the standard injections at 0.025 μmol/mL, 0.1 μmol/mL, and 0.25 μmol/mL, and linear regression was performed.

Sample analysis. Quantitation was carried out with both UV and fluorescence data, using the relative area (area ratio relative to the internal standard). The concentration of each amino acid was calculated in each sample using the linear fitted standard curve with the following formula: concentration of amino acid (μmol/mL)=m×(area or relative area)+b; where m is the slope of the linear fitted curve and b is the Y-intercept of the linear fitted curve. The coefficient of determination should be no less than 0.99.

Calculation of total GRGDSP. The total molar concentration of GRGDSP in a hydrolyzed sample may be calculated by averaging the molar concentrations of each amino acid using the following formula:

Concentration of total GRGDSP in sample (μmol/mL)=(([glycine]/2)+[arginine]+[aspartic acid]+[serine]+[proline])/5.

The concentration of total GRGDSP in the GRGDSP-conjugated alginate (μmol/g) may then be calculated with the formula: (conc. of total GRGDSP in sample (μmol/mL)×(0.25 mL/1.0 mL)×20 mL)/mass of GRGDSP-conjugated alginate (g).

### Example 3. Determining Free Cell-Binding Peptide (CBP) in a CBP-Modified Alginate Sample

To determine the amount of non-conjugated cell-binding peptide (CBP), or “free CBP”, in a sample of CBP-modified polymer (e.g., CBP-modified alginate), the free CBP can be separated from the CBP-modified polymer and quantified. Here, the free CBP was separated from a sample of CBP-modified alginate and quantified using the following procedures.

Sample Preparation. A sample of GRGDSP-modified alginate (1000±50 mg) in saline was weighed into a tube fitted with a molecular weight cut-off (MWCO) filter. Saline (4 mL) was then added to the MWCO tube, and the mixture was inverted and vortexed until the solution was mixed well. The MWCO tube was then centrifuged at 5000 revolutions per minute (rpm) for 90 minutes. After centrifugation, the supernatant above the MWCO filter containing the GRGDSP-conjugated alginate was removed from the tube and discarded. The sample from the bottom of the MWCO tube containing free (non-conjugated) GRGDSP was then collected completely and placed in a 5 mL volumetric flask. The volumetric flask was then brought to volume with water and inverted to mix well. The sample was stored in a scintillation vial at 2-8° C. Aliquots of the sample were analyzed using high pressure liquid chromatography (HPLC).

Preparation of a 1.7 μmol/mL (1 mg/mL) stock standard solution. 50 mg of GRGDSP peptide (standard) was weighed into a scintillation vial, and LCMS-grade water (10 mL) was then added. The vial was mixed by shaking and vortexing to dissolve the peptide completely. The solution was then transferred to a 50 mL volumetric flask, and the vial was rinsed twice with LCMS-grade water and transferred to the volumetric flask using the same pipet to ensure complete transfer of material. The 50 mL volumetric flask was then brought to volume with LCMS-grade water and mixed well by inverting to achieve a stock standard solution at a concentration of 1 mg/mL. The sample was stored at 2-8° C. until use.

Preparation of an 8.5×10⁴ μmol/mL (0.0005 mg/mL) stock standard solution. A 25 μL aliquot of the 1.7 μmol/mL (1 mg/mL) stock standard solution was pipetted into a 50 mL volumetric flask. The volumetric flask was then brought to volume with LCMS-grade water and mixed well by inverting to achieve a stock standard solution at a concentration of 0.0005 mg/mL. The sample was stored at 2-8° C. until use.

HPLC conditions. HPLC was performed with an Agilent 1260 LC with SQ MS, using an XSelect Peptide HSS T3 C18, 2.5 μm, 4.6×100 mm (Waters 186008763) column and equipped with an API-ES positive MS detector (SIM: 588.30). The mobile aqueous phase used was 0.05% trifluoroacetic acid (TFA), and the mobile organic phase was 0.05% TFA in acetonitrile, at a flow rate of 1.0 mL/min. The column run was at ambient temperature. Samples were injected at a volume of 10 μL. First, two blank sample of water were injected, followed by five replicates of the 8.5×10⁻⁴ μmol/mL standard solution. Then, samples containing free GRGDSP were injected in sequence, and every 10 samples were bracketed with an injection of the standard solution. The HPLC sequence was ended by first injecting water, then the 8.5×10⁻⁴ μmol/mL standard, followed by a flush with 50/50 water-acetonitrile. Exemplary HPLC chromatograms can be found in FIGS. 2A-B, showing the chromatograms obtained from the standard solution (FIG. 2A) and sample solution (FIG. 2B).

System suitability criteria. For data to be considered the results were confirmed against the following suitability criteria: no significant interferences in blanks; no more than 2% relative standard deviation (RSD) of retention time in the first 5 injections of the GRGDSP standard; no more than 20% RSD of area in the first 5 injections of the GRGDSP standard; no more than 2% RSD of retention time in the initial 5 injections and all bracketing injections of the GRGDSP standard solution; and no more than 20% RSD of area in the initial 5 injections and all bracketing injections of the GRGDSP standard solution.

HPLC data analysis and calculations. The GRGDSP peak was identified by matching the retention time of the GRGDSP peak in selected ion monitoring (SIM) chromatogram with the standard. The concentration of standard solution (μmol/mL) was first established, and was calculated using the following formula: purity (%)/(100×weight (mg))/50 mL/587.59 g/mol/(1000 mg/g×1,000,000 mol/mol)/2000; where the molecular weight of GRGDSP is 587.59 g/mol, and 2000 is the total dilution factor.

The concentration of free GRGDSP in the samples (μmol/mL) was then determined from the HPLC results and the concentration calculated above, using the formula: area (sample)/(area (standard)×concentration (standard)).

Based on the calculations above, the concentration of free GRGDSP in the modified alginate sample (μmol/g) could then be simply determined. This calculation is represented by the following formula: concentration of free GRGDSP in the modified alginate=(concentration of free GRGDSP in sample (μmol/mL)×5 mL)/weight of conjugate (g).

### Example 4. Determining Conjugated Cell-Binding Peptide (CBP) in a CBP-Modified Alginate Sample

When the amount of free CBP and total CPB (free CPB+conjugated CPB) in a sample is known, the amount of conjugated CBP may be calculated. In cases where the amount of free CBP is less than 2% relative to the CBP, the amount of conjugated CPB may be reported as equal to the value of total CBP. Otherwise, the amount of conjugated CBP may be calculated by subtracting the amount of free CBP (e.g., calculated in Example 3) from the amount of total CBP (e.g., calculated in Example 3). This calculation can be represented by the formula: conjugated CBP (μmol/g)=total CBP (μmol/g)−free CBP (μmol/g).

Converting amount of conjugated CBP in saline solution to the equivalent solid sample. The amount of conjugated CBP in a saline solution of a modified polymer (e.g., CBP-modified alginate) can be converted to the corresponding amount of conjugated CBP of its equivalent form as lyophilized solid, using a simple conversion. This conversion requires determining the concentration of the modified polymer (e.g., CBP-modified alginate) in the saline solution sample, which can be obtained following the protocol outlined in Example 5. Then, simply dividing the amount of conjugated CBP obtained for the saline solution by the concentration of the modified polymer in the saline solution (divided by 100), one may derive the amount of conjugated CBP in the equivalent solid sample. This calculation is represented by the formula: conjugated CBP in solid sample (μmol/g)=conjugated CBP in saline (μmol/g)/(concentration of modified alginate in saline (% w/w)/100).

For example, a GRGDSP-conjugated alginate in saline solution was calculated to have 0.36 μmol/g total GRGDSP, and <0.004 μmol/g free GRGDSP. As the amount of free GRGDSP was less than 2%, the amount of conjugated GRGDSP in the saline sample was determined to be 0.36 μmol/g (the same as total GRGDSP). The concentration of the modified alginate in the saline sample was determined to be 1.39% w/w (following the protocol in Example 5). The calculated amount of conjugated CBP of the corresponding solid sample was then calculated to be 25.90 μmol/g (0.36 μmol/g/[1.39 w/w %/100]=25.90 mol/g).

### Example 5. Determining the Concentration of Modified Polymers in Solution Using Refractive Index

Refractive index (RI) was used in order to determine the concentration of modified polymers (e.g., CBP-modified alginates) in solution without removing the polypeptide (e.g., CBP) from the polymer via, e.g. acid hydrolysis. Here, the concentrations of CBP-modified alginates were determined using a refractometer following one of two protocols outlined below.

Standard Preparation. Approximately 550 mg of the lyophilized solid form of a GRGDSP-modified alginate was weighed into a scintillation vial, and the actual weight (Ws) was recorded. A sterile saline solution was then added in an amount necessary to make a final concentration of 3.0%, and the actual total weight (Wt) was recorded. The mixture was then mixed gently with a tube rotator at 20 rpm for at least 2 hours, and then stored at 2-8° C. overnight to ensure sufficient dissolution. The next day, the sample was warmed to room temperature and again gently mixed for at least 4 hours on a tube rotator at 20 rpm. The resulting 3.0% stock solution should be homogenous without any solid residue.

Dilutions. A series of dilutions was carried out to prepare approximately 5 g solutions, at the target concentrations of 2.5%, 2.0%, 1.5%, and 1.0%. For each concentration, the appropriate amount of the 3.0% stock solution prepared above was transferred to a vial, and the actual weight (Ws) was recorded. Then approximately 5000 mg of sterile saline solution was added, and the actual total weight (Wt) was recorded. Each vial was then gently mixed on a tube rotator at 20 rpm for at least an hour, and stored at 2-8° C. The actual weights, concentrations, and refractive index obtained for each solution is provided in Table 6 below.

TABLE 6
Actual weights, concentrations, and refractive
index (nD) of standard solutions.

| | | Actual | | |
|---|---|---|---|---|
| | Actual | Total | | |
| Nominal | Weight | Weight | Actual | Refractive |
| conc. | (Ws) | (Wt) | conc. | Index |
| (% w/w) | (mg) | (mg) | (% w/w) | (nD) |
| 3.0 | 547.52 (solid) | 18551.49 | 2.951353234 | 1.3390 |
| 2.5 | 4152.90 (3%) | 4888.89 | 2.507046557 | 1.3383 |
| 2.0 | 3296.94 (3%) | 4851.15 | 2.005799559 | 1.3376 |
| 1.5 | 2465.11 (3%) | 4916.60 | 1.479764547 | 1.3367 |
| 1.0 | 1655.26 (3%) | 4864.70 | 1.004225739 | 1.3360 |

Refractometer measurement. At least 5 drops of the sample (at ambient temperature) were added to the clean and dry prism of a LAXCO RBD-5001 refractometer, such that the sample completely covered the prism bottom, and air bubbles on the prism were broken or removed. Measurements were obtained at a temperature of 20° C. Blank measurements were obtained by rinsing the prism twice with saline solution, and measuring the 2^{nd} saline rinse, which had an nD of 1.3343-1.3347. All measurements were obtained in triplicate.

Exemplary test sequence. The measurement of a series of samples was carried out with the following protocol. A saline solution was measured, followed by measuring the standard solution. Then, a first sample was measured in triplicate, followed by each subsequent sample in triplicate. Finally, the standard was measured again, followed by saline. Between every measurement, the prism was rinsed with saline.

Standard calibration curve. The refractive index (nD) measured for each of the standard solutions prepared above (the 3.0%, 2.5%, 2.0%, 1.5%, and 1.0% standard solutions) were plotted with against the actual concentrations (shown in in Table 6), and linear regression was performed to obtain the slope and Y-intercept.

Suitability criteria. The refractive index reading of 0.9% saline should be within 1.3343-1.3347; the calculated concentration should be within 90% to 110% of the theoretical concentration.

Standard curve extrapolation. Concentrations of samples were determined in one of two ways. In the first method, the concentration of each GRGDSP-modified alginate was determined by extrapolation from the standard curve. For example, concentrations of samples were calculated as follows: concentration of modified alginate (% w/w)=m×refractive index+b; where m is the slope of the linear fitted curve and b is the Y intercept. The coefficient of determination was no less than 0.95.

Determination of dn/dc: In a second method, concentrations of GRGDSP-modified alginates in solution were determined by first calculating a specific refractive index increment (i.e., dn/dc value). A dn/dc value was calculated for each of the peptide GRGDSP and a glucuronic acid moiety by first determining the actual concentration (μmol/g) or (% w/w) of each component (GRGDSP or glucuronic acid) using the actual Ws and Wt, then plotting against a corrected refractive index value. The dn/dc value is meant to be a constant for each component at a specific wavelength and temperature. For example, standard concentrations were quantified as follows;

Cone,stnd(μmol/g)=Ws (mg)/MW (g/mol)/Wt (mg)*1,000,000; or

Conc,stnd (% w/w)=Ws (mg)/Wt (mg)*100

The corrected refractive index values were then calculated as follows:

RI, stnd−RI, saline=dn/dC*Conc, stnd, where, differential RI (dn/dC) is the slope of the linear fitted curve.

Exemplary do/dc values are summarized in Table 7.

TABLE 7
Determination of dn/dc values

| | MW | dn/dc | Dn/dc |
|---|---|---|---|
| Component | (g/mol) | (g/mol) | (1/(% w/w)) |
| Glucuronic | 194.14 | 0.00002771 | 0.00142711 |
| acid (GA) | | | |
| GRGDSP | 587.60 | 0.00011501 | 0.00174782 |

Sample analysis: In order to calculate the total concentration of a GRGDSP-modified alginate in solution in this method, the refractive index value for a sample was measured (RI, sample) and used in the following equation to determine the GRGDSP-modified alginate concentration:

Concentration of unmodified alginate (% w/w or mol/g)=[((RI,sample)−(RI,saline)−(dn/dc,GRGDSP))×(concentration,GRGDSP)]/(dn/dc,GA)

For example, concentration of a GRGDSP-modified alginate determined by RI is shown below:

TABLE 8
Exemplary calculation of CBP-modified polymer concentration

| | | | | | % Conj | % Conj |
|---|---|---|---|---|---|---|
| | | | | | (relative | (relative |
| | | | | | to modified | to unmodified |
| | | | | | alginate, | alginate, |
| | Total | Residual | Conj | Conc | weight | molar |
| Description | (saline) | free | (saline) | (Alginate) | ratio) | ratio) |
| GRGDSP | 0.36 | <0.004 | 0.36 | 74.29 | N/A | 0.36/74.29*100 = |
| conjugate | μmol/g | μmol/g | μmol/g | μmol/g | | 0.48% |
| | | (LT 2% | (same as | | | |
| | | total) | total) | | | |
| | 0.02% | | 0.02% | 1.45% | =0.02/ | N/A |
| | w/w | | w/w | w/w | (0.02 + | |
| | | | (same as | | 1.45)*100 = | |
| | | | total) | | 1.36% | |

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A method of evaluating a polymer composition comprising a polymer modified with a polypeptide, the method comprising:
(a) subjecting the polymer composition to reaction conditions that allow for:
(i) release of the polypeptide from the modified polymer; and
(ii) hydrolysis of the polypeptide into component amino acids;
(b) acquiring a value for the concentration of each component amino acid of the polypeptide; and
(c) using the value obtained in step (b), acquiring a value for the concentration of the polypeptide bound to the modified polymer,
thereby evaluating the polymer composition.

2. The method of claim 1, wherein the polymer in the modified polymer is a polysaccharide.

3. The method of claim 2, wherein the polymer in the modified polymer is an alginate.

4. The method of claim 3, wherein the modified alginate has an average molecular weight of 75 kD to 150 kD.

5. The method of claim 3, wherein the alginate has a guluronate to mannuronate (G:M) ratio of greater than or equal to 1.5.

6. The method of claim 1, wherein the polypeptide is covalently bound to the polymer (e.g., alginate).

7. The method of claim 1, wherein the polypeptide is non-covalently bound to the polymer (e.g., alginate).

8. The method of claim 1, wherein the polypeptide is covalently bound to the polymer (e.g., alginate) through a linker (e.g., an amino acid linker).

9. The method of claim 8, wherein the linker comprises at least one glycine residue (e.g., at least 2, 3, or 4 glycine residues).

10. The method of claim 1, wherein the polymer composition or comprises a single type of modified polymer (e.g., modified alginate).

11. The method of claim 1, wherein the polymer composition comprises a plurality of modified polymers (e.g., at least two modified polymers, at least three modified polymers).

12. The method of claim 1, wherein the polypeptide comprises a cell-binding polypeptide (CBP).

13. The method of claim 12, wherein the CBP comprises a sequence selected from RGD, RGDSP, DGEA, FYFDLR, PHSRN, YIGSR, and a sequence listed in Table 1.

14. The method of claim 13, wherein the CBP comprises a plurality of sequences selected from RGD, RGDSP, DGEA, FYFDLR, PHSRN, YIGSR, and a sequence listed in Table 1.

15. The method of claim 6, wherein the polypeptide covalently bound to the modified polymer (e.g., modified alginate) is a linker-CBP.

16. The method of claim 15, wherein the linker-CBP comprises GRGD or GRGDSP.

17. The method of claim 1, wherein the reaction conditions in step (a) comprise contacting the polymer composition with an acidic solution (e.g., an HCl solution, e.g., a 1N-8N HCl solution).

18. The method of claim 1, wherein the reaction conditions in step (a) comprise heating the polymer composition (e.g., at a temperature greater than about 25° C., 40° C., 60° C., 80° C., 100° C., 120° C., or higher).

19. The method of claim 1, wherein the reaction conditions in step (a) comprise exposing the polymer composition to microwave irradiation.

20. The method of claim 1, wherein step (b) comprises a separation step.

21. The method of claim 20, wherein the separation step comprises chromatography (e.g., size-exclusion chromatography, ion-exchange chromatography, gel filtration chromatography, reversed-phase chromatography, or hydrophobic interaction chromatography).

22. The method of claim 1, wherein acquiring a value for the concentration in step (b) comprises determining the area of a chromatogram peak for each component amino acid of the polypeptide.

23. The method of claim 22, wherein acquiring a value for the concentration in step (b) further comprises comparing the area of a chromatogram peak for each component amino acid of the polypeptide with a standard, e.g., to determine the concentration of each component amino acid of the polypeptide.

24. The method of claim 23, wherein the standard comprises a mixture of amino acids.

25. The method of claim 1, further comprising modifying the individual amino acids prior step (b), e.g., prior to chromatographic separation.

26. The method of claim 25, wherein modifying the individual amino acids comprises derivatization with a derivatizing agent.

27. The method of claim 26, wherein the derivatizing agent comprises ortho-phthaladehyde (OPA) or 9-fluoroenylmethyl chloroformate (Fmoc).

28. The method of claim 1, further comprising acquiring a value for the concentration of free polypeptide (i.e., unconjugated polypeptide) in the polymer composition.

29. The method of claim 28, wherein acquiring a value for the concentration of free polypeptide (i.e., unconjugated polypeptide) in the polymer composition comprises:
(a′) separating the polymer composition into a polymer bound fraction and a non-polymer bound fraction;
(b′) retaining the non-polymer bound fraction;
(c′) acquiring a value for the concentration of the polypeptide in the non-polymer bound fraction,
thereby acquiring a value for the concentration of free polypeptide (i.e., unconjugated polypeptide) in the polymer composition.

30. The method of claim 29, wherein the separating of step (a′) comprises filtration, e.g., with a molecular weight cutoff filter.

31. The method of claim 29, wherein step (c′) comprises a separation step.

32. The method of claim 31, wherein the separation step comprises chromatography (e.g., size-exclusion chromatography, ion-exchange chromatography, gel filtration chromatography, reversed-phase chromatography, or hydrophobic interaction chromatography).

33. The method of claim 29, wherein acquiring a value for the concentration of step (c′) further comprises determining the area of a chromatogram peak for polypeptide of the non-polymer bound fraction.

34. The method of claim 29, wherein acquiring the concentration of step (c′) further comprises comparing the area of a chromatogram peak for polypeptide of the non-polymer bound fraction with a standard, e.g., to determine the concentration of polypeptide of the non-polymer bound fraction.

35. The method of claim 34, wherein the standard comprises a single peptide or single polypeptide.

36. The method of claim 1, wherein evaluating a polymer composition comprises determining the concentration of polypeptide conjugated (e.g., covalently bound) to a modified polymer in the polymer composition.

37. The method of claim 1, wherein each polymer in the polymer composition does not comprise an amide bond (e.g., each polymer is not a peptide, polypeptide, or protein).

38. The method of claim 36, wherein determining the concentration of polypeptide conjugated (e.g., covalently bound) to a modified polymer in the polymer composition comprises:
(a″) acquiring a value for the total concentration of polypeptide in the polymer composition or semi-permeable device;
(b″) acquiring a value for the concentration of free polypeptide (i.e., unconjugated polypeptide) in the polymer composition;
(c″) subtracting the value of the concentration of free polypeptide (e.g., as determined in step (b″)) from the value of the total concentration of polypeptide in the polymer composition;
thereby determining the concentration of polypeptide conjugated (e.g., covalently bound) to a modified polymer in the polymer composition.

39. A method of evaluating a polymer composition comprising an alginate modified with a cell-binding polypeptide (CBP), the method comprising:
(a) subjecting the polymer composition to reaction conditions that allow for:
(i) release of the CBP from the modified alginate; and
(ii) hydrolysis of the CBP into component amino acids;
(b) acquiring a value for the concentration of each component amino acid of the CBP; and
(c) using the value obtained in step (b), acquiring a value for the concentration of the CBP bound to the modified alginate,
thereby evaluating the polymer composition.

40. The method of claim 39, wherein the alginate in the modified alginate has an average molecular weight of 75 kD to 150 kD.

41. The method of claim 39, wherein the alginate has a guluronate to mannuronate (G:M) ratio of greater than or equal to 1.5.

42. The method of claim 39, wherein the CBP is covalently bound to the modified alginate, e.g., through a linker (e.g., an amino acid linker).

43. The method of claim 42, wherein the linker comprises at least one glycine residue (e.g., at least 2, 3, or 4 glycine residues).

44. The method of claim 39, wherein the CBP comprises a sequence selected from RGD, RGDSP, DGEA, FYFDLR, PHSRN, YIGSR, and a sequence listed in Table 1.

45. The method of claim 39, wherein the CBP comprises a plurality of sequences selected from RGD, RGDSP, DGEA, FYFDLR, PHSRN, YIGSR, and a sequence listed in Table 1.

46. The method of claim 39, wherein the CBP comprises GRGD or GRGDSP.

47. A method of determining the concentration of a cell-binding polypeptide (CBP) bound to a polymer, the method comprising:
(a) acquiring a value for the concentration of the total CBP in the polymer composition;
(b) acquiring a value for the concentration of the free (i.e., unconjugated) CBP in the polymer composition;
(c) subtracting the value of the concentration of free CBP (e.g., as determined in step (b)) from the value of the total CBP concentration in the polymer composition,
thereby determining the concentration of CBP conjugated (e.g., covalently bound) to a polymer in the polymer composition.

48. The method of claim 47, wherein step (a) is performed prior to step (b).

49. The method of claim 47, wherein step (b) is performed prior to step (a).

50. The method of claim 47, wherein step (a) comprises:
(i) subjecting the polymer composition to reaction conditions that allow for:
(I) release of the CBP from the polymer; and
(II) hydrolysis of the CBP into component amino acids;
(ii) acquiring a value for the concentration of each component amino acid of the CBP; and
(iii) using the value obtained in step (ii), acquiring a value for the concentration of the CBP bound to the modified polymer.

51. A method of evaluating a polymer composition comprising a polymer modified with a polypeptide, the method comprising:
(a) acquiring a value for the refractive index of the polymer composition;
(b) acquiring a value of the total polypeptide conjugated (e.g., covalently bound) to the modified polymer in the polymer composition;
(c) using the values obtained in each of steps (a) and (b), acquiring a value for the concentration of the polymer modified with the polypeptide;
thereby evaluating the polymer composition.

52. The method of claim 51, wherein the polymer in the modified polymer is a polysaccharide.

53. The method of claim 52, wherein the polymer in the modified polymer is an alginate.

54. The method of claim 53, wherein the alginate has an average molecular weight of 75 kD to 150 kD.

55. The method of claim 52, wherein the alginate has a guluronate to mannuronate (G:M) ratio of greater than or equal to 1.5.

56. The method of claim 51, wherein the polypeptide is covalently bound to the polymer (e.g., alginate).

57. The method of claim 51, wherein the polypeptide is non-covalently bound to the polymer (e.g., alginate).

58. The method of claim 51, wherein the polypeptide is covalently bound to the polymer (e.g., alginate) through a linker (e.g., an amino acid linker).

59. The method of claim 58, wherein the linker comprises at least one glycine residue (e.g., at least 2, 3, or 4 glycine residues).

60. The method of claim 51, wherein the polypeptide comprises a cell-binding polypeptide (CBP).

61. The method of claim 60, wherein the CBP comprises a sequence selected from RGD, RGDSP, DGEA, FYFDLR, PHSRN, YIGSR, and a sequence listed in Table 1.

62. The method of claim 60, wherein the CBP comprises a plurality of sequences selected from RGD, RGDSP, DGEA, FYFDLR, PHSRN, YIGSR, and a sequence listed in Table 1.

63. The method of claim 56, wherein the polypeptide covalently bound to the modified polymer (e.g., modified alginate) is a linker-CBP.

64. The method of claim 63, wherein the linker-CBP comprises GRGD or GRGDSP.

65. The method of claim 51, wherein acquiring the value of the refractive index of step (a) comprises acquiring a refractometer reading (nD) at a specific wavelength and/or specific temperature.

66. The method of claim 51, wherein acquiring a value of the total polypeptide conjugated (e.g., covalently bound) to a modified polymer in the polymer composition of step (b) comprises:
(b′) subjecting the polymer composition to reaction conditions that allow for:
(i) release of the polypeptide from the modified polymer; and
(ii) hydrolysis of the polypeptide into component amino acids
(b″) acquiring a value for the concentration of each component amino acid of the polypeptide; and
(b′″) using the value obtained in step (b″), acquiring a value for the concentration of the polypeptide bound to the modified polymer.

67. The method of claim 51, further comprising acquiring a dn/dc value for a component of the polymer composition (e.g., a component of the modified polymer).

68. The method of claim 67, further comprising using the dn/dc value in step (c) of the method, e.g., to acquire a value for the concentration of the polymer modified with the polypeptide

69. A method of determining the concentration of a cell-binding polypeptide (CBP) bound to a polymer (i.e., a CBP-modified polymer) in a polymer composition, the method comprising:
(a) acquiring a value for the refractive index of the polymer composition;
(b) acquiring a value of the total CBP conjugated (e.g., covalently bound) to the polymer in the polymer composition;
(c) using the values obtained in each of steps (a) and (b), acquiring a value for the concentration of the CBP-modified polymer;
thereby determining the concentration of a cell-binding polypeptide (CBP) bound to the polymer in a polymer composition.

70. The method of claim 69, wherein the polymer is an alginate.

71. The method of claim 70, wherein the alginate has an average molecular weight of 75 kD to 150 kD.

72. The method of claim 69, the CBP is covalently bound to the modified alginate, e.g., through a linker (e.g., an amino acid linker).

73. The method of claim 72, wherein the linker comprises at least one glycine residue (e.g., at least 2, 3, or 4 glycine residues).

74. The method of claim 69, wherein the CBP comprises a sequence selected from RGD, RGDSP, DGEA, FYFDLR, PHSRN, YIGSR, and a sequence listed in Table 1.

75. The method of claim 69, wherein the CBP comprises a plurality of sequences selected from RGD, RGDSP, DGEA, FYFDLR, PHSRN, YIGSR, and a sequence listed in Table 1.

76. The method of claim 69, wherein the CBP comprises GRGD or GRGDSP.

77. The method of any one of claims 1, 39, 47, 51, and 69, wherein the level of free polypeptide is less than 0.01 umol/g, e.g., as determined by % weight of polymer in the composition.

78. A polymer composition comprising a polymer modified with a polypeptide, wherein the concentration of the polypeptide is between 0.1 umol/g and 1.0 umol/g, e.g., as determined by % weight of polymer in the composition, e.g., based on a method of any one of claims 1, 39, 47, 51, and 69.

79. A polymer composition comprising a polymer modified with a CBP, wherein the concentration of the CBP is between 0.1 umol/g and 1.0 umol/g, e.g., as determined by % weight of polymer in the composition, e.g., based on a method of any one of claims 1, 39, 47, 51, and 69.

80. A polymer composition comprising an alginate polymer modified with a CBP (e.g., RGD, RGDSP, DGEA, FYFDLR, PHSRN, YIGSR, GRGDSP, and a sequence listed in Table 1), wherein the concentration of the CBP is between 0.1 umol/g and 1.0 umol/g, e.g, as determined by % weight of alginate polymer in the composition, e.g., based on a method of any one of claims 1, 39, 47, 51, and 69.

81. An alginate polymer modified with a CBP, wherein the concentration of the CBP is between 0.1 umol/g and 1.0 umol/g, e.g., as determined by % weight of alginate polymer in the composition, e.g., based on a method of any one of claims 1, 39, 47, 51, and 69.

82. An alginate polymer modified with a CBP selected from RGD, RGDSP, DGEA, FYFDLR, PHSRN, YIGSR, GRGDSP, and a sequence listed in Table 1, wherein the concentration of the CBP is between 0.1 umol/g and 1.0 umol/g, e.g., as determined by % weight of alginate polymer in the composition, e.g., based on a method of any one of claims 1, 39, 47, 51, and 69.
